(19) United States
(12) Patent Application Publication
(10) Pub. No.: US 2025/0281038 A1
(43) Pub. Date: Sep. 11, 2025

(54) SYSTEMS AND METHODS FOR CALIBRATING SPECTRAL DEVICES

(71) Applicant: RETISPEC INC., Toronto (CA)

(72) Inventors: Eliav Shaked, Toronto (CA); Alon Hazan, Toronto (CA); Tommaso Alterini, Toronto (CA)

(73) Assignee: RETISPEC INC., Toronto, ON (CA)

(21) Appl. No.: 19/170,737

(22) Filed: Apr. 4, 2025

Related U.S. Application Data
(63) Continuation of application No. PCT/IB2023/000599, filed on Oct. 9, 2023.
(60) Provisional application No. 63/414,125, filed on Oct. 7, 2022.

(51) Int. Cl. A61B 3/00; A61B 3/12; A61B 3/14

(52) U.S. Cl. CPC A61B 3/0016; A61B 3/12; A61B 3/14; A61B 2560/0223

(57) ABSTRACT

A method includes receiving, by one or more processors, operating characteristics at which a spectral sensor was configured while the spectral sensor captured a multi-dimensional spectral data package of a target object. The method, also, includes identifying, by the one or more processors, one or more artifacts caused by the operating characteristics at which the spectral sensor was configured while the spectral sensor captured the multi-dimensional spectral data package of the target object, and generating, by the one or more processors, a calibrated multi-dimensional spectral data package of the target object by removing the one or more artifacts from the multi-dimensional spectral data package of the target object.

### CROSS-REFERENCE TO RELATED APPLICATIONS

This application is a bypass continuation application of PCT International Application No. PCT/IB2023/000599, filed Oct. 9, 2023, which claims the benefit of and priority to U.S. Provisional Patent Application No. 63/414,125, filed Oct. 7, 2022, the contents of each of which are incorporated herein by reference in their entireties.

### FIELD

This disclosure relates to systems and methods for calibrating spectral systems, such as for example, systems and methods for monitoring spectral devices.

### BACKGROUND

Ophthalmologists examine the eye to provide eye care. To examine the eye, ophthalmologists often use complex optical equipment with a series of adjustable lenses to capture images of the eye. Such optical equipment can introduce artifacts into the resulting images, which may impede the use of those images for diagnostic purposes.

### SUMMARY

The present disclosure relates to a method, including: receiving, by one or more processors, operating characteristics at which a spectral sensor was configured while the spectral sensor captured a multi-dimensional spectral data package of a target object; identifying, by the one or more processors, one or more artifacts caused by the operating characteristics at which the spectral sensor was configured while the spectral sensor captured the multi-dimensional spectral data package of the target object; and generating, by the one or more processors, a calibrated multi-dimensional spectral data package of the target object by removing the one or more artifacts from the multi-dimensional spectral data package of the target object.

In some embodiments, the present disclosure relates to a method, wherein the target object is an eye. In some embodiments, the present disclosure relates to a method, wherein the operating characteristics include an optical configuration of the spectral sensor while the spectral sensor captured the multi-dimensional spectral data package of the target object. In some embodiments, the present disclosure relates to a method, wherein the optical configuration of the spectral sensor includes one or more properties of one or more optical components operably coupled to the spectral sensor. In some embodiments, the present disclosure relates to a method, wherein the one or more properties include at least one of a focal plane, a focal length, a magnification, a distance between optical components, a working distance, an index of refraction, or a direction of a light path. In some embodiments, the present disclosure relates to a method, wherein the operating characteristics include an illumination power at which the spectral sensor captured the multi-dimensional spectral data package of the target object. In some embodiments, the present disclosure relates to a method, wherein the operating characteristics include: an optical configuration of the spectral sensor while the spectral sensor captured the multi-dimensional spectral data package of a target object; and an illumination power at which the spectral sensor captured the multi-dimensional spectral data package of the target object. In some embodiments, the present disclosure relates to a method, wherein receiving the operating characteristics includes receiving the operating characteristics from one or more characteristics sensors coupled to one or more optical components operably coupled to the spectral sensor. In some embodiments, the present disclosure relates to a method, wherein receiving the operating characteristics includes receiving the operating characteristics from one or more characteristics sensors coupled to one or more adjustable controllers of one or more optical components operably coupled to the spectral sensor. In some embodiments, the present disclosure relates to a method, wherein receiving the operating characteristics includes receiving the operating characteristics from one or more characteristics sensors coupled to a light source that illuminates the target object for imaging. In some embodiments, the present disclosure relates to a method, wherein identifying the one or more artifacts includes: identifying, by the one or more processors, a reference multi-dimensional spectral data package captured, by the spectral sensor, of a reference object, wherein the operating characteristics at which the spectral sensor was configured while the spectral sensor captured the reference multi-dimensional spectral data package of the reference object correspond to the operating characteristics at which the spectral sensor was configured while the spectral sensor captured the multi-dimensional spectral data package of the target object; and identifying, the one or more artifacts in the reference multi-dimensional spectral data package of the reference object. In some embodiments, the present disclosure relates to a method, wherein generating the calibrated multi-dimensional spectral data package of the target object includes removing the one or more artifacts identified in the reference multi-dimensional spectral data package of the reference object from the multi-dimensional spectral data package of the target object.

The present disclosure relates to a method including: receiving, by one or more processors, a plurality of operating characteristics at which a spectral sensor was configured while the spectral sensor captured a respective plurality of reference multi-dimensional spectral data packages of a reference object; identifying, by the one or more processors, in each of the plurality of reference multi-dimensional spectral data packages, one or more artifacts caused by each of the plurality of operating characteristics at which the spectral sensor was configured while the spectral sensor captured each of the plurality of the multi-dimensional spectral data package of the reference object; receiving, by one or more processors, operating characteristics at which the spectral sensor was configured while the spectral sensor captured a multi-dimensional spectral data package of a target object; selecting, by the one or more processors, from the plurality of reference multi-dimensional spectral data packages, a reference multi-dimensional spectral data package of the reference object, wherein the operating characteristics at which the spectral sensor was configured while the spectral sensor captured the reference multi-dimensional spectral data package of the reference object corresponds to the operating characteristics at which the spectral sensor was configured while the spectral sensor captured the multi-dimensional spectral data package of the target object; and modifying, by the one or more processors, the multi-dimensional spectral data package of the target object with the reference multi-dimensional spectral data package of the reference object to remove, from the multi-dimensional spectral data package of the target object, one or more artifacts caused by the operating characteristics at which the spectral sensor was configured while the spectral sensor captured the multi-dimensional spectral data package of the target object.

In some embodiments, the present disclosure relates to a method, wherein the target object is an eye. In some embodiments, the present disclosure relates to a method, wherein the operating characteristics include an optical configuration of the spectral sensor while the spectral sensor captured the multi-dimensional spectral data package of the target object and the reference multi-dimensional spectral data package of the reference object. In some embodiments, the present disclosure relates to a method, wherein the optical configuration of the spectral sensor includes one or more properties of one or more optical components operatively coupled to the spectral sensor. In some embodiments, the present disclosure relates to a method, wherein the one or more properties include at least one of a focal plane, a focal length, a magnification, a distance between optical components, a working distance, an index of refraction, or a direction of a light path. In some embodiments, the present disclosure relates to a method, wherein the operating characteristics include an illumination power at which the spectral sensor captured the multi-dimensional spectral data package of the target object and the reference multi-dimensional spectral data package of the reference object.

The present disclosure relates to a method, including: receiving, by one or more processors, environmental conditions at which a spectral sensor captured a multi-dimensional spectral data package of a target object; identifying, by the one or more processors, one or more artifacts caused by the environmental conditions at which the spectral sensor captured the multi-dimensional spectral data package of the target object; and generating, by the one or more processors, a calibrated multi-dimensional spectral data package of the target object by removing the one or more artifacts from the multi-dimensional spectral data package of the target object.

The present disclosure relates to a system, including: an illumination source configured to illuminate an object; a spectral sensor configured to capture a multi-dimensional spectral data package of the object; and one or more processors, the one or more processors being programmed to: receive operating characteristics at which the spectral sensor was configured while the spectral sensor captured a multi-dimensional spectral data package of a target object; identify one or more artifacts caused by the operating characteristics at which the spectral sensor was configured while the spectral sensor captured the multi-dimensional spectral data package of the target object; and generate a calibrated multi-dimensional spectral data package of the target object by removing the one or more artifacts from the multi-dimensional spectral data package of the target object.

In some embodiments, the present disclosure relates to a system, wherein the target object is an eye. In some embodiments, the present disclosure relates to a system, wherein the operating characteristics include an optical configuration of the spectral sensor while the spectral sensor captured the multi-dimensional spectral data package of the target object. In some embodiments, the present disclosure relates to a system, wherein the optical configuration of the spectral sensor includes one or more properties of one or more optical components operably coupled to the spectral sensor. In some embodiments, the present disclosure relates to a system, wherein the one or more properties include at least one of a focal plane, a focal length, a magnification, a distance between optical components, a working distance, an index of refraction, or a direction of a light path. In some embodiments, the present disclosure relates to a system, wherein the operating characteristics include an illumination power at which the spectral sensor captured the multi-dimensional spectral data package of the target object. In some embodiments, the present disclosure relates to a system, wherein the operating characteristics include: an optical configuration of the spectral sensor while the spectral sensor captured the multi-dimensional spectral data package of the target object; and an illumination power at which the spectral sensor captured the multi-dimensional spectral data package of the target object. In some embodiments, the present disclosure relates to a system, further including one or more characteristics sensors coupled to one or more optical components operably coupled to the spectral sensor, wherein the one or more processors are programmed to receive the operating characteristics from the one or more characteristics sensors. In some embodiments, the present disclosure relates to a system, further including one or more characteristics sensors coupled to one or more adjustable controllers of one or more optical components operably coupled to the spectral sensor, wherein the one or more processors are programmed to receive the operating characteristics from the one or more characteristics sensors. In some embodiments, the present disclosure relates to a system, further including one or more characteristics sensors coupled to the illumination source, wherein the one or more processors are programmed to receive the operating characteristics from the one or more characteristics sensors. In some embodiments, the present disclosure relates to a system, wherein to identify the one or more artifacts, the one or more processors are further programmed to: identify a reference multi-dimensional spectral data package captured, by the spectral sensor, of a reference object, wherein the operating characteristics at which the spectral sensor was configured while the spectral sensor captured the reference multi-dimensional spectral data package of the reference object correspond to the operating characteristics at which the spectral sensor was configured while the spectral sensor captured the multi-dimensional spectral data package of the target object; and identify the one or more artifacts in the reference multi-dimensional spectral data package of the reference object. In some embodiments, the present disclosure relates to a system, wherein to generate the calibrated multi-dimensional spectral data package of the target object, the one or more processors are further programmed to remove the one or more artifacts identified in the reference multi-dimensional spectral data package of the reference object from the multi-dimensional spectral data package of the target object. In some embodiments, the present disclosure relates to a system, wherein the spectral sensor is optically coupled to a fundus camera.

The present disclosure relates to a system, including: an illumination source configured to illuminate an object; a spectral sensor configured to capture a multi-dimensional spectral data package of the object; and one or more processors, the one or more processors being programmed to: receive a plurality of operating characteristics at which the spectral sensor was configured while the spectral sensor captured a respective plurality of reference multi-dimensional spectral data packages of a reference object; identify, in each of the plurality of reference multi-dimensional spectral data packages, one or more artifacts caused by each of the plurality of operating characteristics at which the spectral sensor was configured while the spectral sensor captured each of the plurality of the multi-dimensional spectral data package of the reference object; receive operating characteristics at which the spectral sensor was configured while the spectral sensor captured a multi-dimensional spectral data package of a target object; select, from the plurality of reference multi-dimensional spectral data packages, a reference multi-dimensional spectral data package of the reference object, wherein the operating characteristics at which the spectral sensor was configured while the spectral sensor captured the reference multi-dimensional spectral data package of the reference object corresponds to the operating characteristics at which the spectral sensor was configured while the spectral sensor captured the multi-dimensional spectral data package of the target object; and modify the multi-dimensional spectral data package of the target object with the reference multi-dimensional spectral data package of the reference object to remove, from the multi-dimensional spectral data package of the target object, one or more artifacts caused by the operating characteristics at which the spectral sensor was configured while the spectral sensor captured the multi-dimensional spectral data package of the target object.

In some embodiments, the present disclosure relates to a system, wherein the target object is an eye. In some embodiments, the present disclosure relates to a system, wherein the operating characteristics include an optical configuration of the spectral sensor while the spectral sensor captured the multi-dimensional spectral data package of the target object and the reference multi-dimensional spectral data package of the reference object. In some embodiments, the present disclosure relates to a system, wherein the optical configuration of the spectral sensor includes one or more properties of one or more optical components operatively coupled to the spectral sensor. In some embodiments, the present disclosure relates to a system, wherein the one or more properties include at least one of a focal plane, a focal length, a magnification, a distance between optical components, a working distance, an index of refraction, or a direction of a light path. In some embodiments, the present disclosure relates to a system, wherein the operating characteristics include an illumination power at which the spectral sensor captured the multi-dimensional spectral data package of the target object and the reference multi-dimensional spectral data package of the reference object. In some embodiments, the present disclosure relates to a system, wherein the spectral sensor is optically coupled to a fundus camera.

### BRIEF DESCRIPTION OF DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1A shows a block diagram of an exemplary ophthalmic spectral system, according to one or more exemplary embodiments.

FIG. 1B shows an example of an ophthalmic spectral system, according to one or more exemplary embodiments.

FIG. 1C shows an example of a lever controller of the ophthalmic system, according to one or more exemplary embodiments.

FIG. 1D shows an example of a knob controller of the ophthalmic spectral system, according to one or more exemplary embodiments.

FIG. 1E shows an example of digital settings of the ophthalmic spectral system, according to one or more exemplary embodiments.

FIG. 1F shows an illustration of an illustrative 3-dimensional spectral data package, according to one or more exemplary embodiments.

FIG. 1G shows an illustration of an illustrative spectropolarimetric data package, according to one or more exemplary embodiments.

FIG. 1H shows an illustration of an example adapter of the ophthalmic spectral system, according to one or more exemplary embodiments.

FIG. 2A shows a block diagram of an ophthalmic spectral system, a reference object, a sensor, and a datastore, according to one or more exemplary embodiments.

FIGS. 2B-2D shows examples of artifacts.

FIG. 3 shows an illustrative flowchart of a method for processing reference spectral data packages, according to one or more exemplary embodiments.

FIG. 4 shows a flowchart of one method for processing spectral data packages of a target object, according to one or more exemplary embodiments.

FIG. 5 shows a block diagram of a computer-based system and platform, according to one or more exemplary embodiments.

While the above-identified drawings set forth some presently disclosed embodiments, other embodiments are also contemplated, as noted in the discussion below. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Described herein are examples of techniques for calibrating an ophthalmic spectral system used for diagnostic imagery of a patient's eye. A spectral imager of an ophthalmic spectral system and the data packages of the spectral imager can be calibrated, which can enable better image capture or image analysis and improve diagnostic image analysis for identifying presence and/or risk of neurodegenerative diseases like Alzheimer's Disease (AD). More particularly, techniques described herein can receive a multi-dimensional spectral data package of a target object. A sensor can provide operating characteristics, such as an optical configuration (e.g. optical properties, such as, but not limited to refractive compensation, focal length, or other settings) and an illumination power with which a spectral sensor or camera was configured while capturing the spectral data package of the target object. The techniques described herein can be used to select a reference data package(s) captured by the spectral sensor with particular operating characteristics (e.g., in a particular optical configuration and/or under a particular illumination power). The multi-dimensional spectral data packages of the target object can be modified with the reference data package(s) to identify, mitigate the effects of, and/or remove from the data packages artifacts caused by the ophthalmic spectral system.

A camera, sensor, or other imaging device including or operatively coupled to optical equipment can be adjusted to selectively be in one of a plurality of operating characteristics. In some embodiments, operating characteristics can include an optical configuration that the camera, sensor, or imaging device and/or the optical equipment operatively coupled to the camera, sensor, or other imaging device is configured at. An optical configuration can generally be understood as being defined by the collection of optical properties of the optical equipment in or operatively coupled to the imaging device when in a particular configuration. That is, in a first optical configuration, the optical equipment may include a first set of optical properties (where the “set” can be understood as one or more optical properties), and in a second optical configuration, the optical equipment may include a second set of optical properties, where at least one optical property in the second set is different from at least one optical property in the first set. Optical properties of the optical equipment of the imaging device that can include, but are not limited to, focal plane, focal lengths of the optical equipment, magnification, internal and external optical conditions, internal distance between optical and mechanical components, working distance, index of refraction of the optics, direction of the light paths, small pupil mode, myopia compensation settings, red filter, any filter, and IR settings. Optical equipment can vary in optical and mechanical properties to compensate for different conditions of the object under examination. When one or more optical properties of the system are varied, corresponding to different optical configurations of the optical equipment, the specific artifacts or optical aberrations that may affect the quality of multi-dimensional data packages obtained with the imaging device may vary between different optical configurations. In some embodiments, operating characteristics can include the illumination power of the sensor, camera, imaging device/system used to generate a data package (of the eye diagnostics or reference object for calibration, for instance).

Systems described herein can include one or more imaging devices capable of collecting information from across the electromagnetic spectrum. In some embodiments, such imaging devices may include a spectral camera or a spectral sensor. The spectral camera can include a spectral sensor. In some embodiments, the spectral camera may be understood to be the spectral sensor, itself. Therefore, the terms spectral camera and spectral sensor may be used interchangeably. In some embodiments, the spectral imaging device, such as a spectral camera or sensor, can be optically connected to a fundus camera. In some embodiments, the spectral imaging device, such as a spectral camera or sensor, can be incorporated into a fundus camera.

In some embodiments, techniques described herein can be used for removing artifacts from spectral data packages. An optical configuration, defined by optical properties, such as but not limited to, refractive compensation and focal length, and/or an illumination power at which a spectral camera was configured when the data package was obtained can be identified. The optical configuration and/or illumination power can be used to identify a reference data package (collected by imaging a reference object, for instance) that was obtained at corresponding operating characteristics (e.g., a corresponding optical configuration and/or corresponding illumination power), and artifacts can be identified using the corresponding reference data package. The techniques described herein can generate a calibrated multi-dimensional spectral data package of the eye by identifying, mitigating the effects of, and/or removing, from the multi-dimensional spectral data package, the artifacts identified using the reference data package.

A patient's eye is linked by the patient's optic nerve directly to the patient's brain. The inventors have recognized and appreciated that, given this direct connection via neurological tissue, a patient's eye can contain indications of a condition of the patient's brain, and determinations regarding the patient's eye can be used to infer a condition of the patient's brain. For example, proteins produced in a patient's brain can migrate from the brain to the fundus of the eye. The inventors have recognized and appreciated that such proteins can be detectable from spectral data packages captured of the patient's eye, such as when the eye is illuminated with light (e.g., visible light of one or more colors, including white light, and/or non-visible light of one or more spectral ranges or any combination of wavelengths) and light reflected, absorbed, transmitted, emitted or otherwise output from the eye is captured with imaging equipment. Such an illumination and imaging of the eye can be non-invasive for a patient and can present relatively low risk of injury for the patient. By analyzing the data related to the spectral data packages, a testing system can determine whether one or more proteins are present or absent in the patient's eye and/or absolute or relative amounts of the protein(s) in the patient's eye. A testing system can then use this information to determine whether the protein(s) are present in the patient's brain, the absolute or relative amount(s) of the protein(s) in the patient's brain, and/or the patient's status with respect to one or more neurodegenerative diseases. Accordingly, by making determinations regarding proteins or protein levels for a patient's eye using spectral data packages of the eye, a patient's status with regard to one or more neurodegenerative diseases can be obtained.

As the eye is an extension of the central nervous system, linked by the optic nerve directly to the brain, many neurogenerative or neurological conditions or diseases affecting the brain can manifest in the eye, such as protein accumulation, changes to the structure of retinal layers, and other changes in chemical composition, structure, and function. Examples of neurological diseases that affect the eye include Alzheimer's disease, Parkinson's disease, Amyotrophic Lateral Sclerosis, Multiple Sclerosis, Prion disease, Motor neuron diseases (MND), Huntington's disease (HD), Spinocerebellar ataxia (SCA), Spinal muscular atrophy (SMA), and cerebral amyloid angiopathy (CAA). By examining the eye to identify physical changes, these and other neurological diseases can be identified early on to improve health outcomes.

For example, proteins produced in the brain as part of Alzheimer's disease progression, such as beta amyloid and tau, migrate from the brain to the fundus of the eye. In individuals with Alzheimer's disease, both the amyloid and tau levels in the brain are elevated prior to the onset of symptoms. The levels of amyloid and tau are correlated, in that subjects who develop AD tend to have biomarker evidence of elevated amyloid deposition biomarkers (which is detected via abnormal amyloid PET scan or low CSF Ab42 or Ab42/Ab40 ratio) as the first identifiable evidence of abnormality, followed by biomarker evidence of pathologic tau (which is detected via CSF phosphorylated tau, and Tau PET). This may be due to amyloid pathology inducing changes in soluble tau release, leading to tau aggregation later. In some embodiments, the systems and methods of the present disclosure can be used to detect, from a scan of the eye, various disease biomarkers, such as, for example, Tau neurofibrillary tangles, Amyloid Beta deposits, soluble Amyloid Beta aggregates, or Amyloid precursor protein, in the brain or the central nervous system.

In some embodiments, the systems and method of the present disclosure can detect biomarkers indicative of tau pathologies or tauopathies, including, without limitation, total (T-tau), Tau PET, and phosphorylated tau (P-tau). In some embodiments, the biomarkers indicative of a Tauopathy include, but are not limited to, phosphorylated paired helical filament tau (pTau), Early Tau phosphorylation, Late Tau phosphorylation, pTaul81, pTau217, pTau231, total Tau, Plasma AB 42/40, Neurofibrillary tangles (NFTs) and aggregation of misfolded tau protein. In some embodiments, neurofilament light protein (NFL), neurofilaments (NFs) or abnormal/elevated neurofilament light protein (NFL) concentration can be detected. In some embodiments, surrogate markers of a neurodegenerative disorder or neuronal injury can be detected, for example, retinal and optic nerve volume loss or other changes, degeneration within the neurosensory retina, and optic disc axonal injury. In some embodiments, an inflammatory response or neuroinflammation can be detected and may be indicative of neurological disease. In some embodiments, such inflammatory response can be detected in the retinal tissue. Examples of such responses include, but are not limited to, retinal microglia activation, degenerating ganglion cells (ganglion neuron degeneration), or astrocyte activation. Other protein aggregates or biomarkers useful in the methods and systems of the present disclosure include alpha synuclein and TDP43 (TAR DNA binding protein—43) and others described, for example, in Biomarkers for tau pathology (Molecular and Cellular Neuroscience, Volume 97, June 2019, Pages 18-33), incorporated herein by reference in its entirety. In some embodiments, the systems and methods of the present disclosure can be used to detect the presence or absence of protein aggregates or biomarkers indicative of one or more neurological diseases in the patient's eye tissue, brain tissue, tissues of the central nervous system, peripheral nervous system, or in the cerebrospinal fluid (CSF) or any other tissue where such formations or their biomarkers occur. In some embodiments, the systems and methods of the present disclosure detect protein aggregates or biomarkers indicative of one or more neurological diseases without using a dye or ligand. In some embodiments, dyes or ligands can be used to assist the presently disclosed methods and systems. In some embodiments, the results of the optical tests can be confirmed using an anatomic MRI, FDG PET, plasma test, and/or CSF total Tau.

The detection of these biomarkers in the eye can be indicative of the presence or absence of these proteins in the brain or the central nervous system and corresponding risk of developing diseases. The eye can be examined using a variety of non-invasive light-based techniques to identify these health conditions because conditions affecting the optic nerve and retina can result in changes that induce different polarization changes in reflected light as a function of wavelength of the light.

In some embodiments, the systems and methods of the present disclosure eliminate or at least reduce imaging artifacts that can be present in spectral data packages captured using ophthalmic devices. These artifacts are created by the light passing through the ophthalmic device's optical and mechanical components or through the eye itself.

Artifacts in ophthalmic imaging devices can be caused by stray light of optics in the detection and illumination optical paths of the imaging devices, which may be shared or not shared between the two paths. For example, when an object interfaces with other objects, the change of refractive index between the objects can generate a reflection. For instance, if the same optical components are used to generate illumination light and detect light output from an eye, for instance, then reflected light generated from an interface between illumination path optical components can end up being detected as stray light that causes artifacts in the data packet. Therefore, the spectral camera can detect light reflected by the optical components, themselves, instead of or in addition to light reflected or otherwise output by the eye being imaged. As another example, the retina may reflect approximately 2% of the illumination light. Optical components with anti-reflective coatings (that reduce stray light) can reduce the reflected light to 1% at the optimized wavelength. Based on the absorbing power of the ocular media of the eye in some spectral region, the light levels collected from the stray light can be comparable or superior to the reflected light of the retina, which can pollute the data by introducing artifacts. Moreover, the Cornea and Crystalline lens might not have anti-reflective coating and would therefore reflect even more, which would cause even more artifacts.

Another cause of artifacts can be optical aberrations and/or distortion. For instance, optical components, such as even the crystalline lens of the eye and the cornea, can cause optical aberrations that can cause differences in optical properties and optical quality within the multi-dimensional data package.

In some embodiments, the artifacts can be caused by the structure or arrangement of specific hardware of the ophthalmic devices, such as a structure or arrangement of optical components, which can at least partially define the optical configuration of the devices. Artifacts can also be caused by a facility in which the spectral camera is installed, as the spectral data packages generated by the device can be affected by environmental conditions of its surroundings, such as humidity light pollution and/or grains of dust.

In some embodiments, the presence, position, and degree of artifacts may change over time. Artifacts may vary as the ophthalmic devices are adjusted during use, such as by changing an optical configuration to change optical properties, such as refractive power or magnification, or changing illumination power of a light source of a device. Artifacts may, also, vary as humidity, dust, or outside light sources are adjusted in the environment of the ophthalmic devices. Other changing factors may, also, result in a variance of artifacts. For example, artifacts can be caused by movements (e.g., of the eye) that can require registration of the image of the eye. In another example, the positioning of the optics inside an ophthalmic imaging system may vary with temperature, which can cause subtle differences in the artifacts resulting from the relative position of the lenses, such as stray light. Accordingly, it may be difficult to predict whether artifacts will arise, where they will arise, and to what extent they arise.

These artifacts could prevent or impede analysis of the diagnostic images, or at least render analysis more difficult, because the artifacts caused by the optics may appear in the spectral data packages and obscure parts of the eye that may be necessary to analyze for diagnosis or make parts of an image indistinct or sufficiently indistinct to prevent or impede analysis. For example, in some cases, artifacts can affect the periphery of the image of the eye.

Complicating matters further, these ophthalmic devices do not make available information on the structure or current arrangement of the devices, such as the devices particular optical configuration (including a current position of the adjustable optical components), illumination power, or the environment within which the devices are operating. For example, the amount of light reflected by optical components can be proportional to the illumination power, so artifacts, such as stray light, can be proportional to illumination power. The shape and the extent of the stray light artifacts can be dependent on the position or arrangement of optical components defining the optical configuration of the ophthalmic imaging system at the moment of the image acquisition, so artifacts can be dependent on the optical configuration.

The inability to determine, for a spectral camera, factors, such as current optical configuration or illumination power, makes it difficult to estimate at a given time what artifacts might arise, which in turn makes it difficult to compensate for the artifacts. As a result, the inventors have recognized and appreciate that use of ophthalmic devices with a spectral camera for diagnostic imagery is difficult.

Conventionally, calibration of spectral imagers is difficult, as a high-reliability calibration would require conducting an evaluation of the spectral camera at each operating characteristic at each location of use. For example, the technician would have to visit the location in which the spectral camera is installed to identify the artifacts or measure the humidity and light pollution. These calibrations are also costly, limiting their availability to health care providers. Moreover, a lack of calibration of the spectral cameras that are installed in different locations results in difficulty inferring spectral data packages of a patient population. For example, spectral cameras can be scattered around the world and while ideally, they would produce the exact same image of the same patient, there is a chance that one of the spectral cameras would be different and fail to produce the same image. As such, spectral cameras might only be used in isolation for a particular patient by a particular health care provider. However, such use in isolation may not be practical or accurate by failing to compare the patient to the patient population, which can lead to detrimental care for the patient.

The inventors have thus recognized and appreciated that conventional approaches to calibrating and maintaining spectral imagers are not able to provide a convenient, consistent, and low-cost way to calibrate multiple spectral cameras such that they can reliably yield information regarding status with respect to neurodegenerative diseases.

Accordingly, described below are techniques for compensation and calibration to mitigate residual artifacts. For example, proper spatial and spectral calibration can help retrieve artifact-less spectral images. Some techniques described herein are for calibration of ophthalmic spectral systems and editing of spectral data packages to remove artifacts and prepare the spectral data packages for diagnostic imagery analysis. Some techniques described herein include obtaining, with a spectral camera, multi-dimensional spectral data packages of a reference object. A sensor coupled to the spectral camera, or operatively coupled to the spectral camera, can provide information on the optical configuration the spectral camera was in and/or the illumination power used while capturing the multi-dimensional spectral data packages of the reference object, and specifically the optical configuration the camera was in and/or illumination power used when each of a plurality of data packages were obtained. This allows for the creation of a set of spectral data packages, each depicting the reference object, that were obtained at operating characteristics of the spectral camera. The optical configuration and/or the illumination power can be recorded and taken into consideration when identifying artifacts. For example, the optical configuration of the ophthalmic device can affect the shape, size, behavior, location, brightness, spectral signature, or measured property such as velocity, activation, modulation, echo response.

Subsequently, techniques described herein can be used to select, for a spectral data package that is obtained (for diagnostic purposes, for instance) and includes artifacts resulting from the camera (e.g., from the optics, as discussed above), one or more reference data packages that correspond to (e.g., match) the operating characteristics (e.g., optical configuration the spectral camera was in and/or the illumination power used while capturing the multi-dimensional spectral data package). The selected reference data package(s) and the obtained multi-dimensional spectral data package can be compared to identify, mitigate the effects of, and/or remove artifacts that are inserted into the spectral data package captured by the spectral system.

Accordingly, in some embodiments, in addition to or as an alternative to capturing calibration data for the ophthalmic spectral system, techniques described herein can be used for removing artifacts from a spectral data package to generate a calibrated spectral data package. The techniques described herein can receive operating characteristics (e.g., optical configuration and illumination power) at which a spectral camera was configured while capturing a multi-dimensional spectral data package of an eye. The techniques can identify artifacts caused by the ophthalmic spectral system at those operating characteristics (e.g., the optical configuration and the illumination power). The one or more operating characteristics can be recorded and taken into consideration when correcting or removing the artifacts. For example, one or more operating characteristics of the ophthalmic device can affect the shape, size, behavior, location, brightness, spectral signature, or measured property such as velocity, activation, modulation, echo response. The operating characteristics can be used to select one or more corresponding reference data packages (e.g., a reference data package generated under similar or the same operating characteristics). The techniques described herein can generate a calibrated multi-dimensional spectral data package of the eye by removing the one or more artifacts from the multi-dimensional spectral data package of the eye.

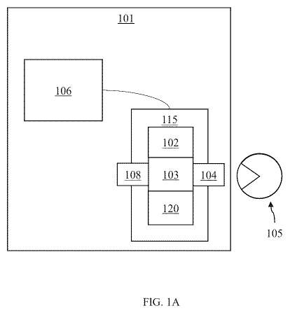

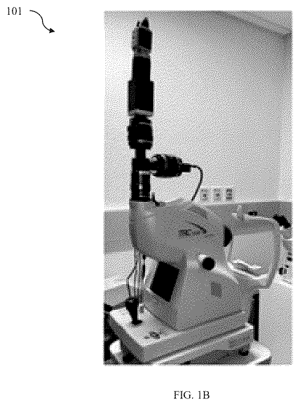

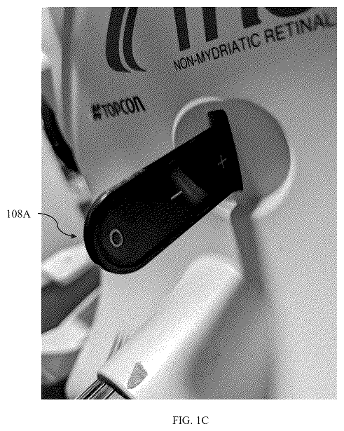

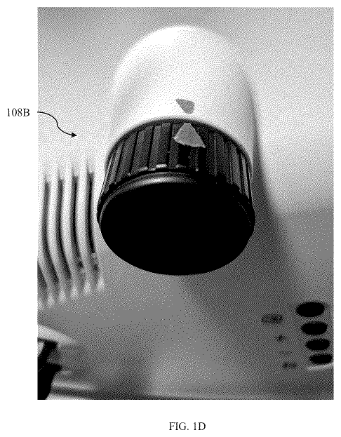

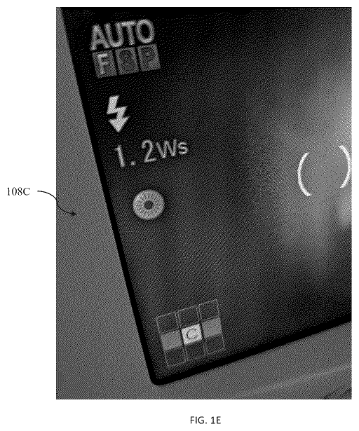

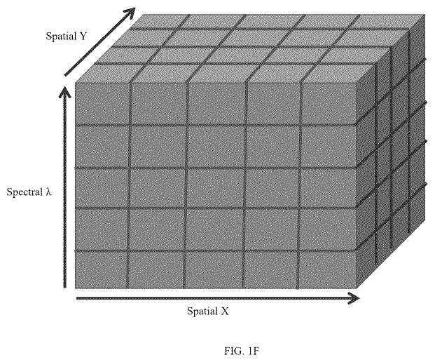

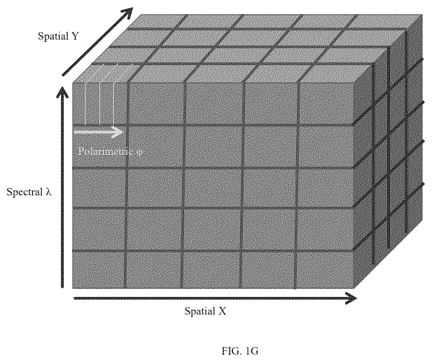

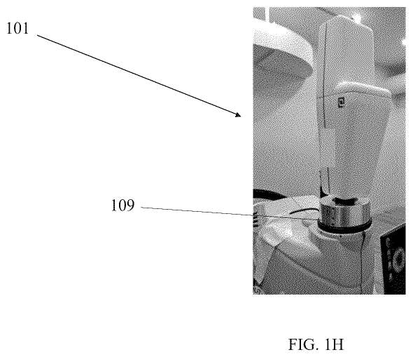

Referring now to FIG. 1A, in some embodiments, the ophthalmic spectral system 101 includes a spectral camera 102, a light source 103 for generating light to pass through an optical system 104 to illuminate an eye 105, where the optical system 104 is configured to receive the light reflected by the eye 105, and a polarizer 120 configured to polarize the light. The ophthalmic spectral system 101 can include imaging equipment and optical components that can introduce artifacts by having a shared or partially shared optical components between the illumination path and the detection path. For example, the ophthalmic spectral system 101 can be any kind of ophthalmic device (e.g., Fundus Camera, Scanning Laser Ophthalmoscope, Optical Coherence Tomography) that has optical components in the illumination path and/or in the detection path and thus can be affected at different levels by stray light. In another example, the ophthalmic spectral system 101 can be a surgical camera, a camera inside a blood vessel, or any other camera for imaging the body.

The spectral camera 102 includes a spectral sensor. In some embodiments, the spectral camera 102 may be understood to be the spectral sensor, itself. Therefore, the terms spectral camera and spectral sensor may be used interchangeably. In some embodiments, the spectral camera 102 can be understood to include a housing component that contains the spectral sensor. The optical system 104 can include one or more optical components that are operatively coupled to the spectral camera 102. In some embodiments, the spectral camera 102 can include the optical system 104. In such embodiments, the optical system 104 can be included in a housing of the spectral camera 102. The optical system 104 can include optical components for directing light toward a target object and collecting light reflected or emitted from the target object. As discussed herein, the optical configuration of the spectral camera 102 can be understood as including the properties, positions, and settings of the optical components of the optical system 104 that is operatively coupled to the spectral camera 102.

In some embodiments, the ophthalmic spectral system 101 includes a computing device 106 configured to receive the one or more spectral data packages, evaluate the spectral data packages, identify one or more biomarkers indicative of a neurodegenerative disease, and determine presence or risk of a neurodegenerative condition. In some embodiments, the spectral camera 102, the light source 103, and the polarizer 120 can be in communication with the computing device 106 for obtaining and analyzing the spectral data packages. The computing device 106 can include one or more processors. The computing device 106 can be programmed to perform any of the methods described herein. The computing device 106 can incorporate the methods discussed herein through software, hardware, and/or firmware. In some embodiments, the computing device 106 includes a control circuit that is a processor. In some embodiments, the control circuit employs firmware. In some embodiments, the control circuit may be a circuit (e.g., controller, processor) that executes instructions, and the instructions may be in the form of firmware or software that controls the systems of the present disclosure.

In some embodiments, the spectral data package can be generated by the spectral camera 102 for analysis by the computing device 106. In some embodiments, the computing device 106 can generate one or more spectral data packages of the eye, receive the one or more spectral data packages of the eye, evaluate the one or more spectral data packages, identify one or more biomarkers indicative of a neurodegenerative pathology in the spectral data package, and determine presence or risk of a neurodegenerative condition based on the biomarkers identified in the spectral data package. In some embodiments, the computing device 106 can identify the pathologies by analyzing the spectral data package generated by the spectral camera 102 of the eye or derived from the image during preprocessing of the spectral data package. In some embodiments, the computing device 106 can calibrate the generated spectral data packages. In some embodiments, the computing device 106 can edit the spectral data packages to remove artifacts and prepare spectral data packages for diagnostic imagery analysis.

In some embodiments, the spectral data package can include imaging data. In some embodiments, the spectral data package can include spatial data, spectral data, structural data, or polarimetric data. In some embodiments, the spectral data package can include spectral data generated by imaging an object (e.g., a retina) at one or more bands of any wavelengths across the electromagnetic spectrum. For example, “spectral data package” may refer to data generated by spectral imaging, such as, for example, hyperspectral, multispectral, or spectropolarimetric imaging. In some embodiments, the spectral imaging can be performed by a RGB sensor, hyperspectral sensor, multispectral sensor, or a spectropolarimetric sensor. For example, the spectral data packages can include spectral information or characteristics related to measurements of the eye at one or more wavelengths. In some embodiments, the computing device 106 can identify the pathologies by analyzing the spectral components in the data package. For example, the computing device 106 can identify the pathologies by analyzing the measurements of the eye at one or more wavelengths.

Referring now to FIG. 1F, in some embodiments, the spectral data package can be a hyperspectral data package visualized as a hyperspectral 3D cube with spatial components. For example, the spectral data package generated by the spectral camera 102 that is a hyperspectral camera can include one or more spatial components and spectral components. In some embodiments, the spectral data package can include a three-dimensional image having a spectral component and a two-dimensional image. In some embodiments, the spectral data package can include a two-dimensional spatial image. In some embodiments, the spectral data package includes a two-dimensional image having a spectral component and a spatial component with a single light intensity value for each image pixel, such as a two-dimensional image generated by a monochrome (grayscale) camera. In some embodiments, the spectral data package includes a two-dimensional image with a single light intensity value for each image pixel, such as a two-dimensional image generated by a monochrome (grayscale) camera. In some embodiments, the spectral data package can comprise a 2-dimensional spatial array in which spatial component is associated with two or more spectral components measured at two or more different wavelengths.

Referring now to FIG. 1G, in some embodiments, the spectral data package can be a spectropolarimetric data package visualized as a spectropolarimetric 4-D cube. The spectral data package can include four-dimensional data or images (4-D image). In some embodiments, the spectral data package can be a 4-D image where the first and second dimensions are x-y dimensions, the third dimension is the spectral λ, and the fourth dimension is the polarization. In some embodiments, the spectral data package includes data elements of (X, Y, λ, φ). In some embodiments, the spectral data package (which can include spectropolarimetric components, spectral-spatio-spectral components, spatial-spectral components, or spatial spectropolarimetric components) can include a spatial X component, a spatial Y component, a spectral λ component of wavelength, and a polarimetric φ component.

In some embodiments, the spectral data package comprises spectropolarimetric components obtained from polarized light reflected from the eye. In some embodiments, the polarimetric component can be polarization angles such as 0°, −45°, 45°, or 90°. In some embodiments, the data package comprises spectral components without polarimetric components. In some embodiments, the spectral data package can include a two-dimensional spatial image with a polarization data package of the light at two or more wavelengths for each image pixel (or a three-dimensional spatial image with a polarization data package of the light at two or more wavelengths for each image voxel). In some embodiments, the 4-D data can be visualized as a 3-D cube noting the 3 dimensions of spatial and spectral (X, Y, λ), where each 3-D voxel of the cube is sliced to the different spectropolarimetric components of the same spatial-spectral position. In some embodiments, the spectral data package can include a 3-dimensional spatial array generated by using a volumetric imaging technique such as optical coherence tomography (OCT). Each element in the spatial array may have arrays of wavelength and polarization values associated with it. In some embodiments, the spectral data package can include depth information based on plenoptic (light field) data packages or time-varying dynamic data packages.

Referring back to FIG. 1A, in some embodiments, the ophthalmic spectral system 101 is a non-invasive ocular light-based system for detecting neurodegenerative disease-associated pathologies in the eye 105. In some embodiments, the ophthalmic spectral system 101 can be used to generate spectral data packages of the eye by providing broadband illumination and imaging optics, including an integrated or external camera to capture the spectral data packages of the fundus of the eye. In some embodiments, the ophthalmic spectral system 101 can provide illumination and spectral data packages of the posterior of the eye (using an internal integrated camera).

The ophthalmic spectral system 101 can be a light-based tool that provides an accessible and non-invasive procedure for identifying at-risk populations of Alzheimer's disease, diagnosis, and tracking treatment and intervention efficacy. The ophthalmic spectral system 101 can be used for optical examination and imaging of part of the fundus, such as the retina to look for signs of AD-associated pathologies in the subject's eye 105 tissue, brain tissue, tissues of the central nervous system, in cerebrospinal fluid (CSF) or any other tissue where such formations or their biomarkers occur. In some embodiments, dyes or ligands can be used to assist with imaging the tissues. In some embodiments, the results of the optical tests can be confirmed using an anatomic MRI, FDG PET, plasma test, and/or CSF total Tau.

Various imaging systems can be employed to gather spectral data. The ophthalmic spectral system 101 of the present disclosure can be presented as a stand-alone imaging system. In some embodiments, the ophthalmic spectral system 101 of the present disclosure can include the spectral camera 102, such as a fundus camera or a similar ophthalmology examination device.

By way of a non-limiting example, FIG. 1B shows a fundus camera for use with the ophthalmic spectral system 101 for capturing one or more spectral image acquisitions of the eye and generating a spectral data package for pathology detection or for diagnosing disease, such as but not limited to Alzheimer's disease, age related macular degeneration, Retinitis Pigmentosa, Glaucoma, Macular Degeneration, Melanoma, Optic nerve drusen, and Retinal Drusen. In some embodiments, the fundus camera can be the spectral camera 102. In some embodiments, the ophthalmic spectral system 101 described herein generates such a spectral data package by using the spectral camera 102 with the fundus camera. In some embodiments, the fundus camera is a Topcon NW8, EX, or DX. In some embodiments, the fundus camera includes an external camera port.

The ophthalmic spectral system 101 and its components (e.g., fundus camera) can be configured at one or more operating characteristics. In some embodiments, the operator can use one or more controllers 108 (e.g., knobs, actuators, switches, buttons) to configure the illumination power and the optical configuration of the ophthalmic spectral system 101 by adjusting one or more properties or positions of one or more optical components of the ophthalmic spectral system 101. For example, the operator can adjust properties or characteristics of the spectral camera 102, the optical element system 104, the light source 103, and the polarizer 120. For example, the operator can use the one or more controllers 108 to adjust the focal length, illumination power, small pupil mode, myopia compensation settings, filters, IR settings, or other optical properties of the ophthalmic spectral system 101 before or while capturing images of the eye. As shown in FIG. 1C, in some embodiments, the controller 108A can be a lever. As shown in FIG. 1D, in some embodiments, the controller 108B can be a knob. For example, the operator can use one or more controllers 108 to adjust position of the optics and thus adjust the refractive power. In another example, the operator can use one or more controllers 108 to adjust the illumination power of the light source 103. In some embodiments, the controllers 108, such as knob(s), can directly control position of the optics of the ophthalmic spectral system 101 (e.g., the optical components operatively coupled to the spectral camera 102), such that as the knob(s) is turned the optics move (e.g., through action of one or more gears or other mechanical elements connected between the knob(s) and the optic(s), or through other mechanisms), and the optics can be continuously adjustable. By being continuously adjustable, the optics can be positioned at any location along a movement path of the optics, rather than only be positioned at discrete positions along the movement path. As shown in FIG. 1E, in some embodiments, the controller 108C can be an interface for selecting digital settings for the ophthalmic spectral system 101. For example, the controller 108C can be a user interface that receives an input from a user and sends instructions to one or more actuators to adjust operating characteristics (e.g., controls a motor coupled to one or more optical components operatively coupled to the spectral camera 102 to adjust the position of the one or more optical components). In some embodiments, the fundus camera might be unable to determine, store, or output the position or setting of the optical components or other operating characteristic at which the fundus camera is configured.

Referring to FIG. 1H, an adapter 109 of the ophthalmic spectral system 101 is depicted. The adapter 109 can be mechanically coupled the fundus camera to one or more other components of the ophthalmic spectral system 101. In some embodiments, the adapter 109 can mechanically couple the spectral camera 102 to one or more other components of the ophthalmic spectral system 101. The adapter 109 can serve not only as a mechanical component to hold the two devices or components together, but it can also include optical properties to determine the precise distance between the optical planes of ophthalmic spectral system 101 or spectral camera 102 (e.g., of the optical components operatively coupled to the spectral camera 102). Through the determined distance, the optical properties of the end-to-end system can be determined, and as such the optical aberrations and artifacts as well. The adapter 109 can be an opto-mechanical component. The adapter 109 can connect components of the ophthalmic spectral system 101 both from a mechanical perspective, to offer a robust grip between the components, and from an optical perspective to maintain a precise positioning of the devices to ensure correct optical configuration and align them around a common optical axis. The adapter 109 can be machined from aluminum or other materials.

Referring back to FIG. 1A, the light source 103 can be configured to illuminate the eye 105. In some embodiments, the light source 103 can be a broadband light source, which emits a wide spectrum of light (e.g., UV, visible, near infrared, and/or infrared wavelength ranges). In some embodiments the light source 103 can be a narrowband light source which emits a narrow spectrum or single wavelength of light. In some embodiments, the light source 103 can emit a single continuous spectrum of light. In some embodiments, the light source 103 can emit a plurality of discontinuous spectra. In some embodiments, the light source 103 can emit light with a constant wavelength range or intensity. In some embodiments, the wavelengths and intensity can be adjustable. In some embodiments, the light source 103 is configured to emit light only at wavelengths relevant for calculating the metrics indicative of disease state. In some embodiments, the light source 103 can comprise one or more LEDs, xenon flash light source, laser or light bulbs, a xenon lamp, a mercury lamp, or any other illuminator or light emitter. The light source 103 can include a single source of light or a combination of multiple sources of light of the same or different types described above.

In some embodiments, the light source 103 generates light having a tunable polarization. In some embodiments, the light source 103 can emit light with circular or linear polarization or having one or more polarization components (e.g., spatial characteristics, frequencies, wavelengths, phases, and polarization states). In some embodiments, the light source 103 can emit light with a random polarization (e.g., partially polarized light that has a random mixture of waves having different spatial characteristics, frequencies, wavelengths, phases, and polarization states).

The polarizer 120 can comprise a polarization filter array comprising one or more polarization filters that let light waves of a specific polarization pass through while blocking light waves of other polarizations. In some embodiments, the polarizer 120 can provide linear, elliptical, or circular polarization. The polarizer 120 can reduce reflections, reduce atmospheric haze, and increase color saturation in the spectral data packages. The polarizer 120 can be an array of polarization filters used to capture and measure different polarizations of incoming light on different pixels at the same time. The filter can provide polarization states at any one or more angles, such as, for example, 0, −45, 45, and 90 degrees. In some embodiments, the polarizer 120 can restrict the polarization of light that illuminates the eye 105 at any given time. In some embodiments, the polarizer 120 is an array of polarization filters each corresponding with one or more pixels of the spectral camera 102. The polarizer 120 can be used to capture and measure different polarizations of incoming light sequentially by allowing light through the polarizer 120. In some embodiments, the polarizer may be combined with or otherwise work in combination with a spectral filter array comprising one or more spectral filters to limit the wavelengths of light received by the spectral cameras 102 to the wavelengths relevant for calculating the metrics indicative of disease state. In some embodiments, the light source 103 includes the polarizer 120 to control or restrict the polarization of light that illuminates the eye 105 and the polarization of light reflected from the eye 105 that is received by the spectral camera 102.

In some embodiments, the polarizer 120 can be placed between the light source 103 and the eye 105. In some embodiments, the polarizer 120 can be placed between the eye 105 and the spectral camera 102. In some embodiments, the polarizer 120 can be placed between both the light source 103 and the eye 105, and another polarizer 120 can be placed between the eye 105 and the spectral camera 102. In some embodiments, the polarizer 120 can be integrated with the light source 103 or with the spectral cameras 102 and in some embodiments, it can be separate. In some embodiments, the polarizer 120 is integrated with the spectral camera 102. In some embodiments, the polarizer 120 can be placed both between the light source 103 and the eye 105, and between the eye 105 and the spectral camera 102.

The spectral camera 102 can be a device or sensor be configured to receive light returned from the eye 105. In some embodiments, the spectral camera 102 can generate one or more spectral data packages based on the light reflected from the eye. In some embodiments, the spectral camera may capture data that comprises spectral, spatial, and polarimetric components from which one or more spectral data packages can be constructed.

The spectral camera 102 may be configured to collect and record spectral data packages reflected from the eye 105, and in some embodiments, the fundus of the eye 105. In some embodiments, the spectral camera 102 can be a spectropolarimetric camera. The light source 103 can direct light toward the eye 105 and the spectral camera 102 can be configured to collect and record light reflected by the eye 105. In some embodiments, the light source 103 can direct the light toward the eye 105 with the same optical assembly configured to collect light reflected from the eye 105. In some embodiments, the light source 103 can direct light toward the eye 105 through a different optical path than is used to collect light reflected from the eye 105. The spectral camera 102 can generate spectral data packages of the eye 105 from light from the light source 103 that is reflected by the eye 105 and received by the spectral camera 102.

In some embodiments, the spectral camera 102 can be a spectral camera, snapshot spectral camera, multispectral camera, spatial camera, or sensor configured to receive light returned from the eye 105 to generate or take one or more spectral data packages of the eye 105, as will be discussed in more detail below. In some embodiments, the spectral camera 102 can be a spectral imaging sensor that can produce or generate the spectral data packages. In some embodiments, in addition to the spectral camera 102, optical coherence tomography (OCT) or confocal scanning laser ophthalmoscopy (SLO) can be used to enhance the spectral data packages. In some embodiments, one or more single photon avalanche detectors (SPADs), photomultiplier tubes (PMTs), or other photon sensing devices can also be used. In some embodiments, the spectral camera 102 includes a spectral sensor. In some embodiments, the spectral sensor can be a snapshot spectral sensor. In some embodiments, the spectral sensor can be a spectral sensor, multispectral sensor, or an RGB sensor. In some embodiments, the spectral sensor can be a Fourier transform spectrometer used with a broadband light source. In some embodiments, any imaging system that allows for the collection of spectral data packages can be used. In some embodiments, the spectral sensor can be a monochromatic sensor or other imaging device used with a tunable light source, and/or multiple light sources of different wavelengths, and/or a broadband light source with spectral filters to generate the spectral components. The spectral camera 102 can be one or more of a microscope (regular or confocal), or optical coherence tomography system which contain spectral cameras 102 (like a camera) configured to receive the spectral data packages and communicate with a computer to transmit the spectral data packages for analysis.

In some embodiments, a plurality of spectral cameras 102 can be used to capture spectral data packages at the same time or in sequence. In some embodiments, the plurality of spectral cameras 102 capture the spectral data packages to identify high spatial resolution and high spectral resolution by using different spectral cameras 102. In some embodiments, a first spectral camera 102 can produce a first spectral data package and a second spectral camera 102 can produce a second spectral data package. In some embodiments, the plurality of spectral cameras 102 capture the spectral data packages so that the spectral data package from a first spectral camera 102 can be analyzed to identify spatial, spectral, or polarization components and determine which second spectral camera 102 should be used and/or which locations or portions of the eye 105 to measure with a second spectral camera 102. In some cases, instead of using a second spectral camera 102, the first spectral camera 102 could be used with different operating characteristics to capture a second spectral data package of the eye 105 with different spatial, spectral, or polarization components. In some embodiments, one or more spectral cameras 102 can include at least two imaging sensors. In some embodiments, a first sensor can create a spatial-spectral image and a second sensor can create a color high-spatial-resolution image.

In some embodiments, the spectral camera 102 comprises a scanning point spectrometer that generates the spectral data packages in two dimensions. In some embodiments, the scanning spectrometer can produce the spectral data packages with both high spatial resolution and high spectral resolution with scanning optics and software. In some embodiments, the spectral camera 102 comprises a line spectrometer that generates the spectral data packages in one dimension (also referred to as a push broom or whisk broom imager). In some embodiments, a line spectrometer can be used to produce a one-dimensional spectral data package with a polarization data package at each wavelength for each pixel along a line without scanning (e.g., 1×N), and a point spectrometer can produce a point ‘image’ (e.g., 1×1) without scanning. In some embodiments, a line spectrometer or point spectrometer can be used to produce higher dimensional spectral data packages with scanning. In some embodiments, the imaging techniques allow the production of three-dimensional spectral data packages in which a spectral data package is produced for each pixel in a three-dimensional volume.

In some embodiments, the spectral camera 102, the light source 103, and the polarizer 120 can be placed inside a housing 115 with optical elements of the optical system 104 that are configured to direct light from the light source 103 to the eye 105 and direct light reflected from the eye 105 to the spectral camera 102. In some embodiments, the element(s) of the ophthalmic spectral system 101 that performs the evaluation of the packages, identification of the biomarkers, and determination of presence or risk of a disease may be integrated with the spectral camera 102 in the same housing 115. In some embodiments, the spectral camera 102, the light source 103, the computing device 106, and the polarizer 120, and/or the optical system 104 can be placed inside the housing 115. In some embodiments, the housing 115 can be a fundus camera. In some embodiments, the spectral camera 102, light source 103, polarizer 120, and/or optical system 104 can be integrated into the housing 115. In some embodiments, the spectral camera 102 can be in the form of a stand-alone device configured to be attached to the housing 115. In some embodiments, the light source 103, optical assembly 104, and/or the polarizer 120 are attached to the ophthalmic spectral system 101. In some embodiments, the light source 103, the spectral camera 102, optical assembly, and/or the polarizer 120 are separate from the housing 115. In some embodiments, the ophthalmic spectral system 101 can further include an array of one or more spectral filters, either integrated with the polarizer 120 or as a standalone component of the ophthalmic spectral system 101. In some embodiments, the element(s) that perform these functions can be separate from the spectral camera 102, such as in a computing device 106 that is outside of the housing 115.

In some embodiments, the ophthalmic spectral system 101 includes a spectral calibration element that can be a light source, an active and/or passive system that can emits, filters, and/or transmits narrowband light at one or more specific wavelengths. In some embodiments, the spectral calibration element can be located within the housing 115 or placed externally to the housing 115. In some embodiments, the spectral calibration element can be coupled to the light source 103. In some embodiments, the spectral calibration element can be adjacent to the light source 103. The computing device 106 can receive a wavelength calibration signal from the spectral cameras 102 that capture the light emitted by the spectral calibration element. The computing device 106 can calculate a pixel to wavelength conversion for spectral data packages from the corresponding wavelength calibration signal. Since the spectral calibration element emits, filters, and/or transmits light at specific wavelengths, the computing device 106 can assign the wavelengths to the pixels or any light collection element or sensor on which that light falls. The computing device 106 can interpolate/extrapolate based on the acquired wavelengths to assign wavelength values to other pixels or any light collection element or sensor.

In some embodiments, the computing device 106 can be configured to obtain, request, or receive a retinal image mosaic comprising the spectral data packages of the eye 105. In some embodiments, the computing device 106 can analyze the one or more spectral data packages to identify biomarkers indicative of a neurodegenerative pathology. In some embodiments, the computing device 106 can generate a digital representation indicative of a presence or absence of the biomarkers in the one or more regions of the eye 105.

In some embodiments, the computing device 106 can regionally segment the spectral data packages to identify pixels in the various components of the eye 105, including the optic disc (nerve head), retina, and fovea. The computing device 106 can identify or determine the existence of one or more AD-associated pathologies, including but not limited to protein aggregates, the protein aggregates including at least one of: Tau neurofibrillary tangles, Amyloid Beta deposits, soluble Amyloid Beta aggregates, or Amyloid precursor protein. In some embodiments, the computing device 106 can use a first imaging modality to identify the locations of blood vessels in the eye 105 (e.g., based on spatial components in an image and/or by detecting blood flow from the image). In some embodiments, the computing device 106 can use a second imaging modality to analyze the spectral components of the blood vessels where the neurological disorders or pathologies may be more likely to be evident. In some embodiments, the computing device 106 can segment the regions within the optic disc to identify more specific components, including a temporal rim, nasal rim, inferior rim, superior rim, and cup regions. In some embodiments, the computing device 106 can perform the segmentation with an automated segmentation algorithm.

The computing device 106 can perform spectral calibration using a previously acquired spectrum of a mercury or mercury-argon lamp, or other light source 103 with well-defined spectral characteristics. The positions of wavelengths of the peaks in a mercury spectrum have well-defined characterized spectra and wavelengths via NIST or other standards. The computing device 106 can compare the wavelengths and the position of the peaks in the mercury or mercury-argon lamp spectrum with the spectrum measured by the spectral camera 102 and the pixels where those wavelengths and the position of those peaks appear in the measured spectrum. The computing device 106 can use the comparison to allow for a pixel to wavelength mapping to be calculated for the spectral data package and the wavelengths of light in subsequent spectral data packages. The pixels in the spectral data packages where the peaks of the mercury lamp are measured can be assigned to the wavelengths of those peaks. By noting the pixels where each of the mercury or mercury-argon lamp peaks is measured, the computing device 106 can calculate an interpolation function to map each spatial pixel to a wavelength value and this interpolation function can be used to correctly assign the wavelength values of each pixel in subsequent spectral data packages.

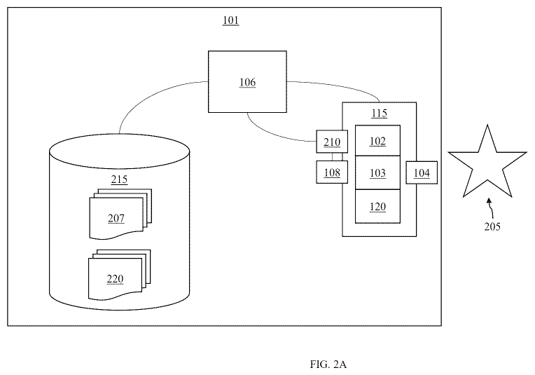

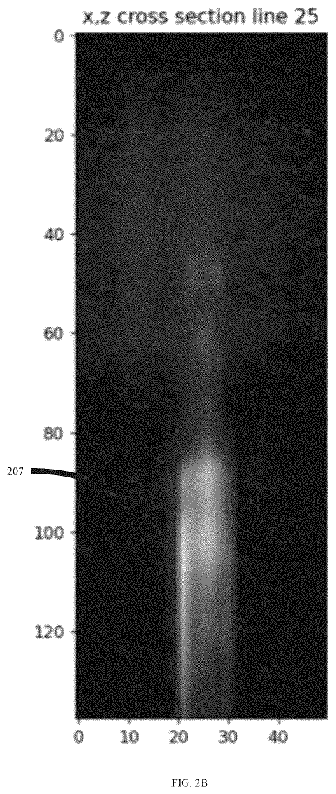

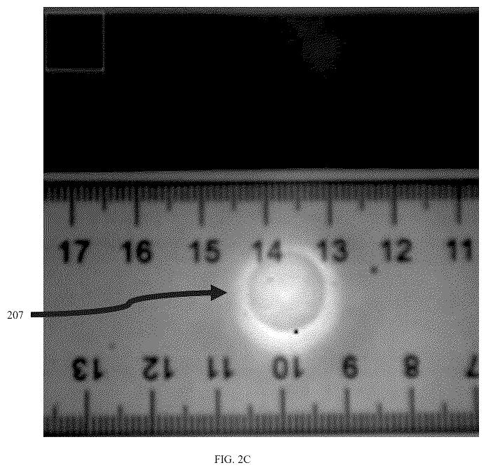

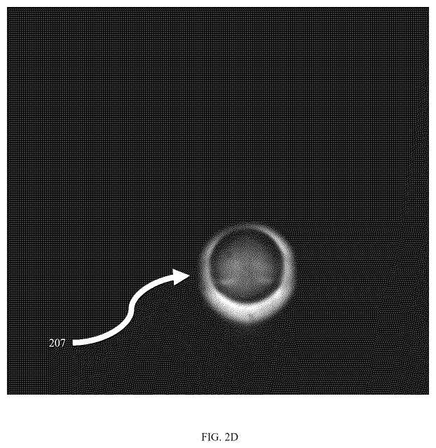

FIG. 2A depicts an embodiment of the ophthalmic spectral system 101 and a reference object 205 for calibration of the ophthalmic spectral system 101 and one or more sensors 210 for sensing one or more operating characteristics, such as an optical configuration and/or an illumination power, of the ophthalmic spectral system 101. It should be appreciated that the ophthalmic spectral system 101 depicted in FIG. 2A can be used to image the eye 105. In other words, the ophthalmic spectral system 101 of FIG. 1A can, also, include the one or more sensors 210, as discussed below. The ophthalmic spectral system 101 can use the one or more operating characteristics when correcting or removing the artifacts 207. For example, one or more operating characteristics of the ophthalmic spectral system 101 can affect the shape, size, behavior, location, brightness, spectral signature, or measured property such as velocity, activation, modulation, echo response. The reference object 205 can be an object designated for use with the ophthalmic spectral system 101 across different calibration iterations, to enable a comparison to a control for calibration purposes. For example, the manufacturing of the reference object 205 can be highly controlled and precise. The reference object 205 can, in some embodiments, mimic the anatomy of the eye 105, while in other embodiments it may have other forms. The reference object 205 can include known reference characteristics. In some embodiments, the reference characteristics can include physical, mechanical, and/or optical properties, such as but not limited to curvatures and materials. In some embodiments, the reference characteristics can include known optical characteristics such as transmission, reflection as function of wavelength, refraction, and modulation. In some embodiments, the “reference object” can refer to an item/article or a volume of space. For instance, in some embodiments, the reference object 205 can be an item that is perfectly black or near perfectly black. Therefore, the reference object 205 will reflect little or no light back to the spectral camera 102. Accordingly, any aberrations or artifacts in the generated spectral data package can be known to be caused by the ophthalmic spectral system 101, itself (e.g. stray light generated within the optical system 104), and not the imaged object (e.g., the reference object 205). As another example, in some embodiments, the reference object 205 can be a black volume of space (e.g., a large, dark, and empty room). Using the ophthalmic spectral system 101 in the black volume of space would result in little to no light being reflected back to the spectral camera. Accordingly, any aberrations or artifacts in the generated spectral data package can be known to be caused by the ophthalmic spectral system 101, itself (e.g. stray light generated within the optical system 104), and not the imaged object (e.g., the reference object 205).

The reference object 205 can be constructed to be scanned or imaged by the ophthalmic spectral system 101. For example, the reference object 205 can be placed in front of the optical element 104. In another example, the reference object 205 can be attached to the ophthalmic spectral system 101 to be scanned by the spectral camera 102.

The spectral camera 102 can generate spectral data packages of the reference object 205. The computing device 106 can cause the spectral camera 102 to capture the spectral data packages of the reference object 205. The computing device 106 can receive the spectral data packages from the spectral camera 102. The computing device 106 can receive and store the spectral data packages. The computing device 106 can compare the reference characteristics of the reference object 205 to the captured spectral data packages to identify artifacts 207 caused by the ophthalmic spectral system 101.

The artifacts 207 can be a signal that is imposed on or included with the imaging data (e.g., spectral data packages of the eye 105 or the reference object 205) but isn't actually present in the object under observation. For example, the artifacts 207 can affect the periphery of the image of the eye 105 or reference object 205. Such signals can be unwanted or undesirable because they can detract from the usability of the imaging data for identifying diseases. For example, the artifact can be caused by stray light, aberration, internal reflection, refraction, and/or background light. Artifacts 207 caused by illumination inhomogeneities of the retinal plane can be corrected by a careful calibration of the imaging field. In some embodiments, the artifacts 207 can be any spectral data that appears in the captured spectral data package of the reference object 205 but is not present in the expected or control spectral data package for such a reference object 205 (e.g., a black image if the reference object 205 is not expected to reflect light).

FIGS. 2B-2D illustrate examples of artifacts 207. FIG. 2B illustrates an example of artifacts 207 by pixel and wavelength band. The horizontal axis (0-40) can describe a pixel location in the image. The vertical axis (0-120) can describe the wavelength band. The artifact 207 can be manifested differently in each pixel and each wavelength band. The artifact 207 can appear everywhere in different brightness, and in particular, the artifact 207 can be concentrated more in pixels 20 to 30 and in spectral channels 80 to 125. FIGS. 2C and 2D illustrate examples of images with artifacts 207. FIG. 2C illustrates an image with the artifact 207 imposed on it. FIG. 2D illustrates an image of an artifact 207 that is isolated.

The computing device 106 can identify the artifacts 207 in the images or spectral data packages. The computing device 106 can generate identifiers of the artifacts 207, which can be used to remove the artifacts 207 from subsequently-captured spectral data packages generated of patients' eyes. For example, the computing device 106 can use information on the detected artifacts 207 identified with the reference object 205 to remove the artifacts 207 from subsequent spectral data packages of the eye 105.

The ophthalmic spectral system 101 can include one or more sensors 210 for sensing the operating characteristics (including optical configuration and/or illumination power) at which the ophthalmic spectral system 101 is configured while generating spectral data packages. For example, the spectral camera 102 might have difficulty identifying, tracking, or recording the one or more operating characteristics, so the sensor 210 can sense such properties. The sensor 210 can recognize the optical configuration at the moment the ophthalmic spectral system 101 generates the spectral data packages. The sensor 210 can recognize the illumination power at the moment the ophthalmic spectral system 101 generates the spectral data packages. In some embodiments, the sensor 210 can measure optical properties such as pupil size and exposure and illumination brightness. In some embodiments, the sensor 210 can measure or detect optical properties such as the focal length and additional lenses or optical components.

In some embodiments, the one or more sensors 210 can measure the back focal length, which is the distance between the back of the last lens and the sensor plane of the ophthalmic spectral system 101. In some embodiments, the one or more sensors 210 can measure the front focal length, which is the distance between the front of the first lens and the object plane of the ophthalmic spectral system 101 or of the eye 105. In some embodiments, the one or more sensors 210 can measure the working distance or eye-relief, which is the distance between the front of the first lens of the ophthalmic spectral system 101 and the first surface of the eye 105. In some embodiments, the one or more sensors 210 can identify the optical configuration of the spectral camera 102 while the while the spectral camera 102 generates spectral data packages.

In some embodiments, the one or more sensors 210 can include a rotary encoder that detects rotation of the one or more controllers 108. For example, the one or more sensors 210 can include a rotary encoder that detects rotation of the controller 108B (e.g., knob or other rotating component). In some embodiments, the one or more sensors 210 can use the rotary encoder to monitor the angle of the focus knob that controls focal length. In some embodiments, the one or more sensors 210 include optical sensors to count the rotations of the one or more controllers 108. For example, the one or more sensors 210 include optical sensors to count the rotations of the controller 108B (e.g., knob). While rotations of the controller 108B have been discussed in detail, it should be appreciated that the one or more sensors 210 can be coupled to any of the above-described controllers to detect movement of the controllers 108, which in turn results in different positioning or setting of the optical elements operatively coupled to the spectral camera 102.

In some embodiments, the one or more sensors 210 can be coupled to the optical elements of the system 104 to identify the optical configuration. In some embodiments, the one or more sensors 210 can be physically coupled (e.g., attached, or glued) to the one or more controllers 108, such as an adjustable knob or a movable piece. For example, the one or more sensors 210 can sense position of the one or more controllers 108 (e.g. an adjustable knob, movable piece, and/or optics), to identify the properties and positions of the optical components, which at least partially define the optical configuration, as they are set by the operator. In some embodiments, the one or more sensors 210 can identify rotations or movement of the one or more controllers 108 as they are moved by an operator or a motor. For example, the one or more sensors 210 can identify rotations of an external manual focusing knob that rotates as the motor moves. In some embodiments, the one or more sensors 210 can be physically coupled (e.g., attached, or glued) to the one or more optical components, themselves, such that the one or more sensors 210 can detect the position or settings of the one or more optical components operatively coupled to the spectral camera 102. In some embodiments, the controller 108C as a digital interface can record the operating characteristics input by the user without the need for the one or more sensors 210. In some embodiments, the one or more sensors 210 can be used to confirm or record the actual operating characteristics after the system adjusts one or more elements in accordance with the instructions received via the controller 108C.

In some embodiments, the one or more sensors 210 can measure the settings, properties, position, and/or movement of the optical components. In some embodiments, the one or more sensors 210 can measure optical properties such as pupil size (e.g., myopia settings, small pupil settings, etc.), exposure, or illumination brightness. In some embodiments, the one or more sensors 210 can measure or detect one or more operating settings such as small pupil mode, myopia compensation settings, red filter, any filter, or IR settings. In some embodiments, the spectral camera 102, with an adjustable focal length, can vary the relative position among optical components operative coupled to the spectral camera 102 to correct for images captured while the spectral camera 102 is not in focus. In some embodiments, the one or more sensors 210 can identify feedback from the motor that is moving the optics. In some embodiments, the one or more sensors 210 can be communicatively coupled (e.g., via a wired or wireless connection, such as USB) to the computing device 106. In some embodiments, the one or more sensors 210 can transmit the measured optical configuration to the computing device 106.

In some embodiments, the one or more sensors 210 can identify the illumination power of the spectral camera 102 or light source 103 while generating the spectral data packages. The one or more sensors 210 can transmit the illumination power absolute or relative value to the computing device 106. The illumination power can be a degree to which the spectral camera 102 converges or diverges light, which can be related to a specific optical configuration. In some embodiments, the one or more sensors 210 can be coupled to the spectral camera 102 or the light source 103 to identify the illumination power. In some embodiments, the one or more sensors 210 include a camera to detect or identify the illumination power, eye (left or right), or anatomical view (Disc, Fovea, etc.). For example, in some embodiments, the one or more sensors 210 can be included in the ophthalmic spectral system 101 that can be a fundus camera that is a closed box.

In some embodiments, the one or more sensors 210 can be printed (e.g., 3D printed) to fit on the spectral camera 102 and/or light source 103 to identify or measure the operating characteristics of the spectral camera 102. For example, the one or more sensors 210 can include a plastic housing that houses the encoder to detect the operating characteristics. In some embodiments, the one or more sensors 210 is a camera positioned to monitor the ophthalmic spectral system 101. In some embodiments, the spectral camera 102, the light source 103, the computing device 106, the polarizer 120, the optical system 104, and the one or more sensors 210 can be placed inside the housing 115. In some embodiments, the ophthalmic spectral system 101 can identify one or more operating characteristics without the one or more sensors 210 because the one or more operating characteristics of the ophthalmic spectral imaging system 101 are already recorded or known.

The ophthalmic spectral system 101 can include a datastore 215 for storing reference data packages 220 of the reference object 205. The datastore 215 can be a database, hard drive, or any other storage system. As shown in FIG. 2A, the datastore 215 can be communicatively coupled to the computing device 106. In some embodiments, the datastore 215 can be communicatively coupled to the spectral camera 102. In some embodiments, the datastore 215 can be communicatively coupled to the light source 103. In some embodiments, the datastore 215 can be communicatively coupled to the one or more sensors 210. In some embodiments, the datastore 215 can be communicatively coupled to one or more of the spectral camera 102, the light source 103, the one or more sensors 210, or the computing device 106. In some embodiments, the spectral camera 102, the light source 103, the computing device 106, the polarizer 120, the one or more sensors 210, and the datastore 215 can be placed inside the housing 115.

The reference data packages 220 can include spectral values of the reference object 205. The reference data packages 220 can include spectral values of the reference object 205 be captured at each of a plurality of optical configurations and a plurality of illumination powers at which the spectral camera 102 can be configured. Each of the reference data packages 220 can indicate the optical configuration and/or illumination power corresponding to the spectral values collected (i.e. the optical configuration and/or illumination power used to collect the spectral values). For example, the reference data packages 220 can include metadata or identifiers that indicate the focal length, the working distance, the refractive power, the illumination power, and any other desirable operating characteristics.

In some embodiments, the computing device 106 can cause the reference data packages 220 to undergo a preprocessing cue, which can include white normalization, dark image subtraction, background image subtraction, and/or artifacts image subtraction. The computing device 106 can acquire the reference data packages 220 after or before the data acquisition, or loaded from the datastore 215, which can include a repository of images acquired during calibration. In some embodiments, the computing device 106 can receive the reference data packages 220 from a network or other storage device (e.g., USB), and transmit the reference data packages 220 for storage in the datastore 215.

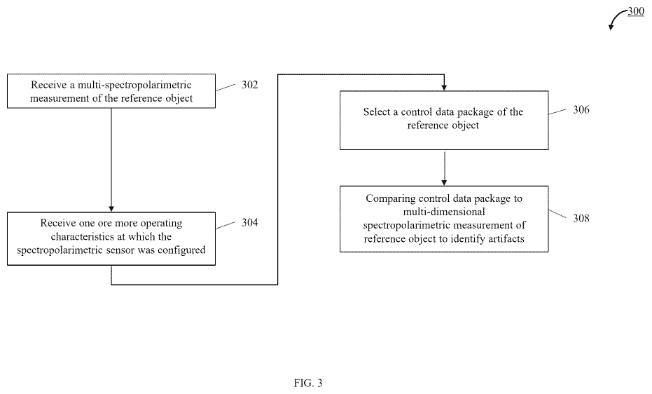

FIG. 3 illustrates a method 300 for obtaining calibration data for the ophthalmic spectral system 101. The method 300 can be performed by the computing device 106, in some embodiments. The method 300 is illustrated in connection with obtaining a single calibration data package but can be performed multiple times to obtain multiple calibration data packages to form a calibration data set.

The method 300 begins at step 302, in which the computing device 106 can operate the spectral camera 102 to obtain, or otherwise receive, a multi-dimensional spectral data package of the reference object 205.

In step 302, the computing device 106 can receive, from the spectral camera 102, a multi-dimensional spectral data package of the reference object 205. Copies of the reference object 205 can be distributed to each ophthalmic spectral system 101 for inter system calibration. For example, before generating spectral data packages of one or more eyes 105 (e.g., at the beginning of a work day, before each patient, hourly, or otherwise periodically at a time interval or occasionally upon occurrence of an event or satisfaction of one or more criteria), the computing device 106 can generate spectral data package of the reference object 205. An operator can place the reference object 205 in front of the optical element 104 for imaging by the ophthalmic spectral system 101. In some embodiments, the computing device 106 can generate the spectral data packages of the reference object 205 by generating spectral data packages of the facility without light (e.g., dark room data packages).

In some embodiments, the computing device 106 can identify the ambient conditions (e.g., temperature or humidity) of the facility, and request spectral data packages of the reference object 205 if the ambient conditions change by more than a threshold. For example, the computing device 106 can cause the spectral camera 102 to capture spectral data packages of the reference object 205 once a year if the ambient conditions are expected to stay constant, or every day if the conditions are expected to vary more frequently.

The computing device 106 can select one or more operating characteristics of the ophthalmic spectral system 101 at which to configure the ophthalmic spectral system 101 while generating the spectral data packages. In some embodiments, the computing device 106 can select the optical configuration of the spectral camera 102. In some embodiments, the computing device 106 can select the illumination power of the spectral camera 102. For example, the computing device 106 can select to configure an optical configuration and/or illumination power of the spectral camera 102. The computing device 106 can cause the light source 103 to illuminate the reference object 205 with light. The computing device 106 can cause the spectral camera 102 to generate the spectral data packages of the reference object 205 that is illuminated by the light source 103. The computing device 106 can receive the spectral data packages generated by the spectral camera 102. In some embodiments, the computing device 106 can identify or receive spectral data packages of different sections of the reference object 205.

At step 304, the computing device 106 can determine, using data provided by the one or more sensors 210, operating characteristics (including optical configuration and/or illumination power) with which the spectral camera 102 was configured when the data package was obtained at step 302 and/or environmental conditions in which the data package was obtained. The operating characteristics with which the spectral camera 102 was configured in step 302 can include characteristics that affect the illumination of the subject of the data package and/or that affect a manner in which the camera 102 acquires light for a data package or captures a data package. For example, the operating characteristics can include different optical properties of a specific optical configuration, the arrangement of the optics of the camera 102 (e.g., the arrangement of the optical elements operatively coupled to the spectral camera), and/or an illumination power of a light source of the camera 102 or ophthalmic spectral system 101 at the time the data package is captured in step 302. In some embodiments, acquiring the operating characteristics can include operating the one or more sensors 210 to detect an operating characteristic(s), such as recognizing the optical configuration and/or illumination power. The computing device 106 of the ophthalmic spectral system 101 can be communicatively coupled to one or more environmental sensors configured to detect one or more environmental conditions, such as the temperature, of the environment that the spectral camera 102 is operating.

At step 304, the computing device 106 can receive, from the one or more sensors 210 coupled to the ophthalmic spectral system 101, the one or more operating characteristics at which the spectral camera 102, or generally the ophthalmic spectral system 101, was configured while capturing the multi-dimensional spectral data packages of the reference object 205.

The one or more operating characteristics with which the ophthalmic spectral system 101 was configured may include characteristics that affect the illumination of the subject of the data package and/or that affect a manner in which the camera 102 acquires light for a data package or captures a data package. For example, one or more operating characteristics of the ophthalmic device can affect the shape, size, behavior, location, brightness, spectral signature, or measured property such as velocity, activation, modulation, echo response.

In some embodiments, the one or more operating characteristics can include an optical configuration of the camera 102 and/or an illumination power of a light source of the camera 102 or ophthalmic spectral system 101, at the time the data package is captured in step 302. In some embodiments, the one or more sensors 210 can receive the optical configuration at which the spectral camera 102 was configured while capturing the multi-dimensional spectral data packages of the reference object 205. The optical configuration of the camera 102 can generally refer to the properties, position, arrangement, and/or settings of the optical elements operatively coupled to the spectral camera 102 (e.g., the optical elements that direct light toward and receive light from the reference object 205).

As mentioned above, while FIG. 3 illustrates obtaining a single data package and determining artifacts related to that data package, embodiments are not so limited. In some embodiments, the computing device 106 can generate spectral data packages of the reference object 205 at a plurality of optical configurations, a plurality of illumination powers, and/or a plurality of other settings or environmental conditions. This might involve performing the method of FIG. 3 multiple times, for different operating characteristics or environmental conditions. In some such embodiments, the computing device 106 can drive the camera to be configured over time with different operating characteristics, such as by driving the optics to different locations and therefore different optical configurations that generate different artifacts. In some embodiments, the one or more sensors 210 can be an element of a drive system that can move optics, and the one or more sensors 210 can detect the optical configuration (or other operating characteristic) as the camera is configured and operating over time. The computing device 106 can receive the operating characteristics (e.g., focal lengths, eye relief or refraction compensation) with which the spectral camera 102 was configured when capturing each data package. In some embodiments, the computing device 106 can receive the optical configuration properties and the illumination power of the spectral camera 102 when capturing each data package. In some embodiments, once the data packages are generated at multiple optical configurations, illumination powers, or other settings, the data packages can be stored for later use in mitigating the effects of artifacts 207 in later-captured data packages of, for example, patients' eyes to be used for diagnostic purposes.

In some embodiments, the computing device 106 can communicate with computing devices associated with other spectral cameras 102 and ophthalmic spectral systems 101 for inter system calibration. For example, each computing device 106 of each ophthalmic spectral system 101 can generate spectral data packages of the reference object 205. The reference object 205 can be a common reference point for each ophthalmic spectral system 101. The computing devices can share the spectral data packages to compare the facilities housing the ophthalmic spectral systems 101.

At step 306, the computing device 106 can select control data packages of the reference object 205. The control data package can be used by the computing device 106 to identify artifacts 207 within the spectral data packages captured in step 302 by comparing the captured data packages to one or more control data packages in which the artifacts 207 would not appear. For instance, in some embodiments, because the reference object 205 is of known properties, a specific control data package, free of any artifacts or aberrations, can be expected to be generated when the reference object 205 is imaged. As an example, based on the properties of the reference object 205, the control data package may be a black image (e.g., when the reference object 205 is expected to return little to no light to the spectral camera 102). At step 308, the computing device 106 can identify one or more artifacts in the multi-dimensional spectral data package of the reference object 205. In some embodiments, the computing device can identify one or more artifacts in the multi-dimensional spectral data package of the reference object 205 by comparing the multi-dimensional spectral data package of the reference object 205 to the one or more selected control data packages of the reference object 205. Because the control data package can be free of any artifacts, a comparison to the multi-dimensional spectral data package of the reference object 205 can identify artifacts in the multi-dimensional spectral data package of the reference object 205 that are a result of the operating characteristics of the spectral camera 102. In some embodiments, where the control data package is a black image, any collected light in the multi-dimensional spectral data package of the reference object 205 can be determined to be an artifact caused by the operating characteristics of the spectral camera 102. In some embodiments, the computing device 106 can identify a position or spectral component of the artifact 207 in the multi-dimensional spectral data package of the reference object 205. In some embodiments, the computing device 106 can identify the attributes or characteristics of the artifacts 207. For example, the computing device 106 can identify a shape or size of the artifact 207 in the spectral data package.

In some embodiments, the computing device 106 can compare the multi-dimensional spectral data package to the control data package by performing a white balancing. In some embodiments, the computing device 106 can divide the multi-dimensional spectral data packages one by one by the control data packages. In some embodiments, the computing device 106 can subtract the control data package from the multi-dimensional spectral data package to identify the artifacts 207. In some embodiments, the computing device 106 can apply mathematical transformations to the multi-dimensional spectral data package and the control data package to identify the artifacts 207. For example, the computing device 106 can apply a Fourier transform to identify artifacts 207 that are phase artifacts or image distortions. In another example, the computing device 106 can apply a multiplication to compensate for artifacts 207 that are transmission artifacts such as single pixel related artifacts, single channel related transmission, absorption, or reflection artifacts, or absorption, transmission and/or reflection artifacts.

As noted above, the method of FIG. 3 can be carried out for a plurality of operating characteristics of the spectral camera 102 (e.g., under a variety of optical configurations, illumination power, and/or environmental conditions). In some embodiments, based on the analysis, the computing device 106 can identify artifacts 207 caused by the spectral camera 102 at each of the one or more operating characteristics at which the spectral camera 102 can be configured. For example, based on the analysis, the computing device 106 can identify artifacts 207 caused by the spectral camera 102 at each of the plurality of focal lengths at which the spectral camera 102 can be configured. In some embodiments, each multi-dimensional spectral data package having identified artifacts can be stored in a database and tagged with operating characteristics that the spectral camera 102 was configured when generating the spectral data package. The database can then be referenced to pull a particular multi-dimensional spectral data package of the reference object 205 for calibration or correction, as described below.

In some embodiments, it can be burdensome to collect a multi-dimensional spectral data package of the reference object 205 under each possible operating characteristics that the spectral camera 102 can be configured. In such embodiments, the computing device 106 can collect a critical mass of multi-dimensional spectral data package of the reference object 205, identify the artifacts in the spectral data packages, and interpolate predicted data packages and associated artifacts in the database. It should be appreciated that the spectral packages stored in the database for later use in calibration and correction, may be corrected themselves. In other words, in some embodiments, the database stores spectral data packages that have been corrected based on the control data package, such that the stored spectral data packages are composed entirely of artifacts caused by the spectral camera 102 operating characteristics and a particular configuration of operating characteristics.

It should be appreciated that the method 300 can be carried out in any order besides that depicted in FIG. 3. For instance, in some embodiments, as a first step, the computing device 106 can receive one or more operating characteristics at which the spectral camera 102 is configured, and in a second step, the computing device can receive the multi-dimensional spectral data package of the reference object 205. It should, also, be appreciated that one or more steps can be carried out substantially simultaneously. For instance, the computing device 106 can simultaneously receive one or more operating characteristics at which the spectral camera 102 is configured and receive the multi-dimensional spectral data package of the reference object 205.

While FIG. 3 has been discussed with particular focus on identifying artifacts caused by the particular operating characteristics of the spectral camera 102 (e.g., the optical configuration and/or illumination power under which the spectral camera 102 collected the multi-dimensional spectral data package of the reference object 205), the same methods can be used to identify artifacts caused by a particular operating environment that the spectral camera 102 was operated in addition to or instead of the determinations relating to operating characteristics.

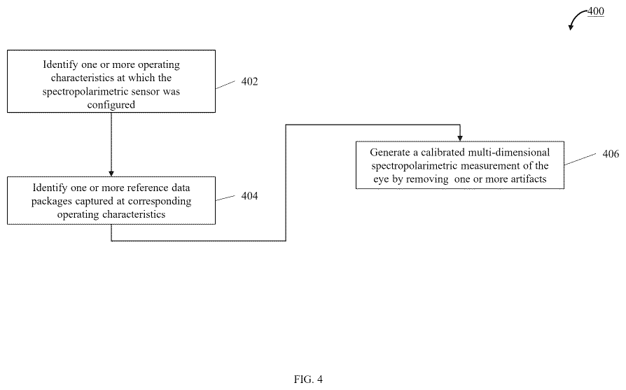

FIG. 4 illustrates a method 400 for editing of a multi-dimensional spectral data package to calibrate the spectral data package by removing artifacts 207 that may have been inserted by the camera system, to prepare spectral data packages of the eye 105 for diagnostic imagery analysis. The method 400 may be performed by the computing device 106. At step 402, the computing device 106 can receive the operating characteristics (e.g., optical configuration and the illumination power) at which the spectral camera 102 was configured while capturing a multi-dimensional spectral data package of the eye 105, or other target object to be analyzed. At step 402, the computing device can receive environmental conditions under which the spectral camera 102 operated while capturing a multi-dimensional spectral data package of the eye 105, or other target object to be analyzed. At step 404, the computing device 106 can identify one or more reference data packages (of the reference object 205, for instance) captured by the spectral camera 102 at operating characteristics (e.g., optical configuration and/or illumination power) corresponding with the operating characteristics of the spectral camera 102 while capturing the multi-dimensional spectral data package of the eye 105. The reference data package(s) can be used in step 404 to identify one or more artifacts 207 present in the data package of the eye 105. At step 406, the computing device 106 can generate a calibrated multi-dimensional spectral data package of the eye 105 by removing the artifacts 207 from the multi-dimensional spectral data package of the eye 105.

At step 402, the computing device 106 can receive (e.g., from the sensor 210) operating characteristics (e.g., optical configuration and/or illumination power) at which the spectral camera 102 was configured while capturing the multi-dimensional spectral data packages of the eye 105. The computing device 106 can cause the light source 103 to illuminate the eye 105 with light. The computing device 106 can cause the spectral camera 102 to generate the spectral data package from a region of the eye 105 that is illuminated by the light source 103. The computing device 106 can receive the spectral data packages generated by the spectral camera 102. In some embodiments, the computing device 106 can identify or receive spectral data packages of regions of the eye 105. The computing device 106 can identify spatial information about a corresponding region in the spectral data packages. The spatial information can comprise texture, formations, and patterns in the corresponding region. In some embodiments, the computing device 106 applies a pixel-wise analysis to the spectral data packages. At step 402, the computing device 106 can receive environmental conditions at which the spectral camera 102 operated while capturing the multi-dimensional spectral data packages of the eye 105.

It should be appreciated that the computing device 106 can receive the operating characteristics before, during, or after the multi-dimensional spectral data packages of the eye 105 is captured. It should be appreciated that the computing device 106 can receive the operating characteristics and/or the environmental conditions in the same manner as described above. That is, the computing device 106 operates substantially the same way to determine operating characteristics and/or environmental conditions while the multi-dimensional spectral data packages of the eye 105 is captured as it does to determine operating characteristics and/or environmental conditions while the multi-dimensional spectral data packages of the reference object 205. Therefore, the various methods and configurations for determining operating characteristics and/or environmental conditions (e.g., receiving data from the one or more sensors 210) are not repeated in detail herein with respect to method 400.

At step 404, the computing device 106 can identify one or more reference data packages (e.g., of the reference object 205) that were obtained by the spectral camera 102 at operating characteristics that correspond to the operating characteristics with which the data package of the eye 105 was obtained in step 402. Such operating characteristics may include the optical configuration and/or illumination power. In addition to or instead of operating characteristics, at step 404, the computing device 106 can identify one or more reference data packages that were obtained at the same environmental conditions as data package of the eye 105. Particularly, in some embodiments, the computing device 106 can access the database of tagged reference data packages discussed with respect to the method 300 to determine a corresponding reference data package (e.g., a reference data package collected under corresponding operating characteristics and/or environmental conditions). In some embodiments, the reference data packages can have the artifacts caused by the corresponding operating characteristics and/or environmental conditions labeled in the reference data packages. In some embodiments, the reference data packages can be corrected against a control data package, as described above, such that the entirety of information contained in the corrected reference data package is one or more artifacts. In some embodiments, a corresponding operating characteristic may be an identical operating characteristic, such as the same optical configuration or same illumination power being used in obtaining the reference data package and the data package of the eye 105. In some embodiments, a corresponding operating characteristic may be the closest match between the operating characteristic for the data package obtained of the eye 105 in step 402 and the operating characteristics available in a reference data set. It should be appreciated that the same embodiments apply to corresponding environmental conditions.

The computing device 106 can use the reference data package(s) to mitigate the impact of or remove one or more artifacts from the data package of the eye 105 obtained in step 402. For example, the computing device 106 can identify the artifacts that were associated with a reference data package when the reference data package was obtained (e.g., in a process akin to that of FIG. 3). Since the reference data package was obtained with corresponding operating characteristics, the artifact(s) present in the reference data package may be the same as or similar to the artifact(s) that may be present in the data package obtained in step 402. Accordingly, the computing device 106 can process the data package of step 402 based on the artifact(s) from the reference data package to remove the artifacts from the data package of the eye 105. In some embodiments, the computing device 106 can process the data package of the eye 105 to locate and subsequently remove the artifacts identified in the corresponding reference data package. In some embodiments, the computing device 106 can directly modify the data package of the eye 105 with the corresponding reference data package, particularly when the reference data package is corrected to only include artifacts. For instance, the computing device 106 can divide the multi-dimensional spectral data package of the eye 105 the corresponding reference data package, or the computing device 106 can subtract the reference data package from the multi-dimensional spectral data package of the eye 105 to remove the artifacts 207 from the data package of the eye 105.

It should be appreciated that the computing device 106 can identify more than one corresponding reference data package. For instance, a first reference data package showing artifacts generated from corresponding operating conditions can be identified, and a second reference data package showing artifacts generated from a corresponding illumination power can be identified. The data package of the eye 105 can then be compared to both corresponding reference data packages to locate and remove artifacts in the data package of the eye 105.

In some embodiments, the computing device 106 can infer the presence of artifacts 207 in the data package of the eye 105 based on multiple reference data packages 220 captured with operating characteristics that do not exactly match the operating characteristics of the data package obtained in step 402, but that correspond to the operating characteristics such as by being within an acceptable range of the operating characteristics of the data package obtained in step 402. For example, the computing device 106 can use a subset of reference data packages 220 to extrapolate to account for other values. In some embodiments, the computing device 106 can use linear interpolation and regression for predicting the intermediate or out-of-range data. For example, if the computing device 106 selects reference data packages 220 for illumination powers (noted here as the energy of the illumination flash) of three Joules, four Joules, and five Joules, the computing device 106 can interpolate intermediate illumination powers (e.g., 3-4 and 4-5 Joules) of illumination power.

In some embodiments, the computing device 106 can identify the artifacts 207 in the data package of the eye 105 based on a back reflection factor that matches properties and parameters of optical configurations. For example, the computing device 106 can identify how much of the light from the light source 103 is reflected in the environment in which the system 101 is maintained. In some embodiments, the computing device 106 can calibrate the back reflection factor. The computing device 106 can use the feedback from the optical configuration sensors and the feedback from the light power to identify the coordinates to the correct back reflection factor and dataset.

In some embodiments, the computing device 106 can tag or register the artifacts 207 in the data package of the eye 105. In some embodiments, the computing device 106 can store the position or value of the artifacts 207 in the spectral data package. In some embodiments, the computing device 106 can tag each artifact 207 with the illumination power level and optical configuration of the system 101 while the artifact 207 was captured. In some embodiments, the computing device 106 can shift (translating and/or rotating using either rigid or elastic transformations) the positions of the artifacts 207 so that the spatial components overlap in a co-registered coordinate system with at least one of the calibrated multi-dimensional spectral data packages. In another example, the computing device 106 can divide the spectral data packages of the eye 105 by the reference data packages 220 of the reference object 205.

At step 406, the computing device 106 can generate calibrated multi-dimensional spectral data packages of the eye 105 by removing the artifacts 207 from the multi-dimensional spectral data packages of the eye 105. In some embodiments, the computing device 106 can remove the artifacts 207 to generate the calibrated multi-dimensional spectral data packages of the eye 105. For example, the computing device 106 can identify spectral components corresponding to the artifacts 207. The computing device 106 can remove the spectral components corresponding to the artifacts 207 to remove the artifacts 207 from the spectral data package of the eye 105 to generate the calibrated multi-dimensional spectral data package of the eye 105.

In some embodiments, the computing device 106 can subtract the spectral components of a reference data package 220 from the measured spectral data package of the eye 105 to remove the artifacts 207 from the data package of the eye 105. In some embodiments, each artifact 207 has the same number of spectral components of a spectral image. The computing device 106 can subtract the spectral components of each artifact 207 from each spectral component of the image of the eye 105. For example, data at positions of an image or other content of a data package that correspond to the artifacts 207 in the reference data package may be subtracted from data at corresponding positions or content of the measured data package of the eye 105. Such a subtraction of data may effect a removal of the artifacts 207 from the image/package of the eye 105.

In some embodiments, the computing device 106 identifies artifacts 207 by using machine learning. It should be appreciated that the computing device 106 can implement the machine learning algorithm by way of neural networks. The machine learning algorithm can include logistic regression, variational autoencoding, convolutional neural networks, transformers, or other statistical techniques used to identify and discern neurodegenerative disease-associated pathologies. The machine learning algorithm can also use spectral scattering models, other scattering models, or optical physics models that are validated a priori. The neural network may comprise a plurality of layers, some of which are defined and some of which are undefined (or hidden). The neural network is a supervised learning neural network.

In some embodiments, the neural network may include a neural network input layer, neural network middle hidden layers, and a neural network output layer. Each of the neural network layers include a plurality of nodes (or neurons). The nodes of the neural network layers are connected, typically in series. The output of each node in a given neural network layer is connected to the input of one or more nodes in a subsequent neural network layer.

Each node is a logical programming unit that performs an activation function (e.g., a transfer function) for transforming or manipulating data based on its inputs, a weight (if any) and bias factor(s) (if any) to generate an output. The activation function of each node results in a particular output in response to particular input(s), weight(s) and bias factor(s).

The inputs of each node may be scalar, vectors, matrices, objects, data structures and/or other items or references thereto. Each node may store its respective activation function, weight (if any) and bias factors (if any) independent of other nodes. In some example embodiments, the decision of one or more output nodes of the neural network output layer can be calculated or determined using a scoring function and/or decision tree function, using the previously determined weight and bias factors.

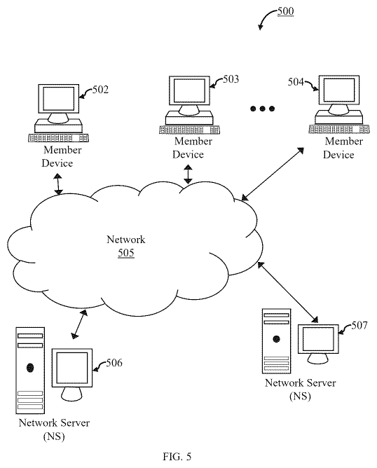

FIG. 5 depicts a block diagram of a computer-based system and platform 500 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 500 may be configured to manage a large number of members and concurrent transactions, as detailed herein. In some embodiments, the exemplary computer-based system and platform 500 may be based on a scalable computer and network architecture that incorporates various strategies for assessing the data, caching, searching, and/or database connection pooling. An example of the scalable architecture is an architecture that is capable of operating multiple servers.

In some embodiments, referring to FIG. 5, member computing device 502, member computing device 503 through member computing device 504 (e.g., clients) of the exemplary computer-based system and platform 500 may include virtually any computing device capable of receiving and sending a message over a network (e.g., cloud network), such as network 505, to and from another computing device, such as servers 506 and 507, each other, and the like. In some embodiments, the member devices 502-504 may be personal computers, multiprocessor systems, microprocessor-based or programmable consumer electronics, network PCs, and the like. In some embodiments, one or more member devices within member devices 502-504 may include computing devices that typically connect using a wireless communications medium such as cell phones, smart phones, pagers, walkie talkies, radio frequency (RF) devices, infrared (IR) devices, CBs, integrated devices combining one or more of the preceding devices, or virtually any mobile computing device, and the like. In some embodiments, one or more member devices within member devices 502-504 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NIOTA, 3G, 4G, 5G, GSM, GPRS, Wi-Fi, WiMAX, CDMA, satellite, Bluetooth, ZigBee, etc.). In some embodiments, one or more member devices within member devices 502-504 may include may run one or more applications, such as Internet browsers, mobile applications, voice calls, video games, videoconferencing, and email, among others. In some embodiments, one or more member devices within member devices 502-504 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as Hypertext Markup Language (HTML), a wireless application protocol (WAP), a Handheld Device Markup Language (HDML), such as Wireless Markup Language (WML), WMLScript, XML, JavaScript, and the like. In some embodiments, a member device within member devices 502-504 may be specifically programmed by either Java, .Net, QT, C, C++ and/or other suitable programming language. In some embodiments, one or more member devices within member devices 502-504 may be specifically programmed include or execute an application to perform a variety of possible tasks, such as, without limitation, messaging functionality, browsing, searching, playing, streaming or displaying various forms of content, including locally stored or uploaded messages, images and/or video, and/or games.

In some embodiments, the exemplary network 505 may provide network access, data transport and/or other services to any computing device coupled to it. In some embodiments, the exemplary network 505 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In some embodiments, the exemplary network 505 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 505 may include and implement, as an alternative or in conjunction with one or more of the above, a WiMAX architecture defined by the WiMAX forum. In some embodiments and, optionally, in combination of any embodiment described above or below, the exemplary network 505 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 505 may be transmitted based at least in part on one of more communication modes such as but not limited to: NFC, RFID, Narrow Band Internet of Things (NBIOT), ZigBee, 3G, 4G, 5G, GSM, GPRS, Wi-Fi, WiMAX, CDMA, satellite and any combination thereof. In some embodiments, the exemplary network 505 may also include mass storage, such as network attached storage (NAS), a storage area network (SAN), a content delivery network (CDN) or other forms of computer or machine-readable media.

In some embodiments, the exemplary server 506 or the exemplary server 507 may be a web server (or a series of servers) running a network operating system, examples of which may include but are not limited to Microsoft Windows Server, Novell NetWare, or Linux. In some embodiments, the exemplary server 506 or the exemplary server 507 may be used for and/or provide cloud and/or network computing. Although not shown in FIG. 5, in some embodiments, the exemplary server 506 or the exemplary server 507 may have connections to external systems like email, SMS messaging, text messaging, ad content providers, etc. Any of the features of the exemplary server 506 may be also implemented in the exemplary server 507 and vice versa.

In some embodiments, one or more of the exemplary servers 506 and 507 may be specifically programmed to perform, in non-limiting example, as authentication servers, search servers, email servers, social networking services servers, SMS servers, IM servers, MMS servers, exchange servers, photo-sharing services servers, advertisement providing servers, financial/banking-related services servers, travel services servers, or any similarly suitable service-base servers for users of the member computing devices 501-504.

In some embodiments and, optionally, in combination of any embodiment described above or below, for example, one or more exemplary computing member devices 502-504, the exemplary server 506, and/or the exemplary server 507 may include a specifically programmed software module that may be configured to send, process, and receive information using a scripting language, a remote procedure call, an email, a tweet, Short Message Service (SMS), Multimedia Message Service (MMS), instant messaging (IM), internet relay chat (IRC), mIRC, Jabber, an application programming interface, Simple Object Access Protocol (SOAP) methods, Common Object Request Broker Architecture (CORBA), HTTP (Hypertext Transfer Protocol), REST (Representational State Transfer), or any combination thereof.

Non-limiting embodiments of the present disclosure are set out in the following clauses:

Clause 1. A method, comprising: receiving, by one or more processors, operating characteristics at which a spectral sensor was configured while the spectral sensor captured a multi-dimensional spectral data package of a target object; identifying, by the one or more processors, one or more artifacts caused by the operating characteristics at which the spectral sensor was configured while the spectral sensor captured the multi-dimensional spectral data package of the target object; and generating, by the one or more processors, a calibrated multi-dimensional spectral data package of the target object by removing the one or more artifacts from the multi-dimensional spectral data package of the target object.

Clause 2. The method of the preceding clause, wherein the target object is an eye.

Clause 3. The method of any preceding clause, wherein the operating characteristics comprise an optical configuration of the spectral sensor while the spectral sensor captured the multi-dimensional spectral data package of the target object.

Clause 4. The method of any preceding clause, wherein the optical configuration of the spectral sensor comprises one or more properties of one or more optical components operably coupled to the spectral sensor.

Clause 5. The method of any preceding clause, wherein the one or more properties comprise at least one of a focal plane, a focal length, a magnification, a distance between optical components, a working distance, an index of refraction, or a direction of a light path.

Clause 6. The method of any preceding clause, wherein the operating characteristics comprise an illumination power at which the spectral sensor captured the multi-dimensional spectral data package of the target object.

Clause 7. The method of any preceding clause, wherein the operating characteristics comprise: an optical configuration of the spectral sensor while the spectral sensor captured the multi-dimensional spectral data package of a target object; and an illumination power at which the spectral sensor captured the multi-dimensional spectral data package of the target object.

Clause 8. The method of any preceding clause, wherein receiving the operating characteristics comprises receiving the operating characteristics from one or more characteristics sensors coupled to one or more optical components operably coupled to the spectral sensor.

Clause 9. The method of any preceding clause, wherein receiving the operating characteristics comprises receiving the operating characteristics from one or more characteristics sensors coupled to one or more adjustable controllers of one or more optical components operably coupled to the spectral sensor.

Clause 10. The method of any preceding clause, wherein receiving the operating characteristics comprises receiving the operating characteristics from one or more characteristics sensors coupled to a light source that illuminates the target object for imaging.

Clause 11. The method of any preceding clause, wherein identifying the one or more artifacts comprises: identifying, by the one or more processors, a reference multi-dimensional spectral data package captured, by the spectral sensor, of a reference object, wherein the operating characteristics at which the spectral sensor was configured while the spectral sensor captured the reference multi-dimensional spectral data package of the reference object correspond to the operating characteristics at which the spectral sensor was configured while the spectral sensor captured the multi-dimensional spectral data package of the target object; and identifying, the one or more artifacts in the reference multi-dimensional spectral data package of the reference object.

Clause 12. The method of any preceding clause, wherein generating the calibrated multi-dimensional spectral data package of the target object comprises removing the one or more artifacts identified in the reference multi-dimensional spectral data package of the reference object from the multi-dimensional spectral data package of the target object.

Clause 13. A method comprising: receiving, by one or more processors, a plurality of operating characteristics at which a spectral sensor was configured while the spectral sensor captured a respective plurality of reference multi-dimensional spectral data packages of a reference object; identifying, by the one or more processors, in each of the plurality of reference multi-dimensional spectral data packages, one or more artifacts caused by each of the plurality of operating characteristics at which the spectral sensor was configured while the spectral sensor captured each of the plurality of the multi-dimensional spectral data package of the reference object; receiving, by one or more processors, operating characteristics at which the spectral sensor was configured while the spectral sensor captured a multi-dimensional spectral data package of a target object; selecting, by the one or more processors, from the plurality of reference multi-dimensional spectral data packages, a reference multi-dimensional spectral data package of the reference object, wherein the operating characteristics at which the spectral sensor was configured while the spectral sensor captured the reference multi-dimensional spectral data package of the reference object corresponds to the operating characteristics at which the spectral sensor was configured while the spectral sensor captured the multi-dimensional spectral data package of the target object; and modifying, by the one or more processors, the multi-dimensional spectral data package of the target object with the reference multi-dimensional spectral data package of the reference object to remove, from the multi-dimensional spectral data package of the target object, one or more artifacts caused by the operating characteristics at which the spectral sensor was configured while the spectral sensor captured the multi-dimensional spectral data package of the target object.

Clause 14. The method of the preceding clause, wherein the target object is an eye.

Clause 15. The method of clause 13 or clause 14, wherein the operating characteristics comprise an optical configuration of the spectral sensor while the spectral sensor captured the multi-dimensional spectral data package of the target object and the reference multi-dimensional spectral data package of the reference object.

Clause 16. The method of any one of clauses 13-15, wherein the optical configuration of the spectral sensor comprises one or more properties of one or more optical components operatively coupled to the spectral sensor.

Clause 17. The method of any one of clauses 13-16, wherein the one or more properties comprise at least one of a focal plane, a focal length, a magnification, a distance between optical components, a working distance, an index of refraction, or a direction of a light path.

Clause 18. The method of any one of clauses 13-17, wherein the operating characteristics comprise an illumination power at which the spectral sensor captured the multi-dimensional spectral data package of the target object and the reference multi-dimensional spectral data package of the reference object.

Clause 19. A method, comprising: receiving, by one or more processors, environmental conditions at which a spectral sensor captured a multi-dimensional spectral data package of a target object; identifying, by the one or more processors, one or more artifacts caused by the environmental conditions at which the spectral sensor captured the multi-dimensional spectral data package of the target object; and generating, by the one or more processors, a calibrated multi-dimensional spectral data package of the target object by removing the one or more artifacts from the multi-dimensional spectral data package of the target object.

Clause 20. A system, comprising: an illumination source configured to illuminate an object; a spectral sensor configured to capture a multi-dimensional spectral data package of the object; and one or more processors, the one or more processors being programmed to: receive operating characteristics at which the spectral sensor was configured while the spectral sensor captured a multi-dimensional spectral data package of a target object; identify one or more artifacts caused by the operating characteristics at which the spectral sensor was configured while the spectral sensor captured the multi-dimensional spectral data package of the target object; and generate a calibrated multi-dimensional spectral data package of the target object by removing the one or more artifacts from the multi-dimensional spectral data package of the target object.

Clause 21. The system of the preceding clause, wherein the target object is an eye.

Clause 22. The system of any preceding clause, wherein the operating characteristics comprise an optical configuration of the spectral sensor while the spectral sensor captured the multi-dimensional spectral data package of the target object.

Clause 23. The system of any preceding clause, wherein the optical configuration of the spectral sensor comprises one or more properties of one or more optical components operably coupled to the spectral sensor.

Clause 24. The system of any preceding clause, wherein the one or more properties comprise at least one of a focal plane, a focal length, a magnification, a distance between optical components, a working distance, an index of refraction, or a direction of a light path.

Clause 25. The system of any preceding clause, wherein the operating characteristics comprise an illumination power at which the spectral sensor captured the multi-dimensional spectral data package of the target object.

Clause 26. The system of any preceding clause, wherein the operating characteristics comprise: an optical configuration of the spectral sensor while the spectral sensor captured the multi-dimensional spectral data package of the target object; and an illumination power at which the spectral sensor captured the multi-dimensional spectral data package of the target object.

Clause 27. The system of any preceding clause, further comprising one or more characteristics sensors coupled to one or more optical components operably coupled to the spectral sensor, wherein the one or more processors are programmed to receive the operating characteristics from the one or more characteristics sensors.

Clause 28. The system of any preceding clause, further comprising one or more characteristics sensors coupled to one or more adjustable controllers of one or more optical components operably coupled to the spectral sensor, wherein the one or more processors are programmed to receive the operating characteristics from the one or more characteristics sensors.

Clause 29. The system of any preceding clause, further comprising one or more characteristics sensors coupled to the illumination source, wherein the one or more processors are programmed to receive the operating characteristics from the one or more characteristics sensors.

Clause 30. The system of any preceding clause, wherein to identify the one or more artifacts, the one or more processors are further programmed to: identify a reference multi-dimensional spectral data package captured, by the spectral sensor, of a reference object, wherein the operating characteristics at which the spectral sensor was configured while the spectral sensor captured the reference multi-dimensional spectral data package of the reference object correspond to the operating characteristics at which the spectral sensor was configured while the spectral sensor captured the multi-dimensional spectral data package of the target object; and identify the one or more artifacts in the reference multi-dimensional spectral data package of the reference object.

Clause 31. The system of any preceding clause, wherein to generate the calibrated multi-dimensional spectral data package of the target object, the one or more processors are further programmed to remove the one or more artifacts identified in the reference multi-dimensional spectral data package of the reference object from the multi-dimensional spectral data package of the target object.

Clause 32. The system of any preceding clause, wherein the spectral sensor is optically coupled to a fundus camera.

Clause 33. A system, comprising: an illumination source configured to illuminate an object; a spectral sensor configured to capture a multi-dimensional spectral data package of the object; and one or more processors, the one or more processors being programmed to: receive a plurality of operating characteristics at which the spectral sensor was configured while the spectral sensor captured a respective plurality of reference multi-dimensional spectral data packages of a reference object; identify, in each of the plurality of reference multi-dimensional spectral data packages, one or more artifacts caused by each of the plurality of operating characteristics at which the spectral sensor was configured while the spectral sensor captured each of the plurality of the multi-dimensional spectral data package of the reference object; receive operating characteristics at which the spectral sensor was configured while the spectral sensor captured a multi-dimensional spectral data package of a target object; select, from the plurality of reference multi-dimensional spectral data packages, a reference multi-dimensional spectral data package of the reference object, wherein the operating characteristics at which the spectral sensor was configured while the spectral sensor captured the reference multi-dimensional spectral data package of the reference object corresponds to the operating characteristics at which the spectral sensor was configured while the spectral sensor captured the multi-dimensional spectral data package of the target object; and modify the multi-dimensional spectral data package of the target object with the reference multi-dimensional spectral data package of the reference object to remove, from the multi-dimensional spectral data package of the target object, one or more artifacts caused by the operating characteristics at which the spectral sensor was configured while the spectral sensor captured the multi-dimensional spectral data package of the target object.

Clause 34. The system of clause 33, wherein the target object is an eye.

Clause 35. The system of clause 33 or clause 34, wherein the operating characteristics comprise an optical configuration of the spectral sensor while the spectral sensor captured the multi-dimensional spectral data package of the target object and the reference multi-dimensional spectral data package of the reference object.

Clause 36. The system of any one of clauses 33-35, wherein the optical configuration of the spectral sensor comprises one or more properties of one or more optical components operatively coupled to the spectral sensor.

Clause 37. The system of any one of clauses 33-36, wherein the one or more properties comprise at least one of a focal plane, a focal length, a magnification, a distance between optical components, a working distance, an index of refraction, or a direction of a light path.

Clause 38. The system of any one of clauses 33-37, wherein the operating characteristics comprise an illumination power at which the spectral sensor captured the multi-dimensional spectral data package of the target object and the reference multi-dimensional spectral data package of the reference object.

Clause 39. The system of any one of clauses 33-38, wherein the spectral sensor is optically coupled to a fundus camera.

The description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments. Embodiment examples are described as follows with reference to the figures. Identical, similar, or identically acting elements in the various figures are identified with identical reference numbers and a repeated description of these elements is omitted in part to avoid redundancies.

From the foregoing description, it will be apparent that variations and modifications may be made to the embodiments of the present disclosure to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method, comprising:
receiving, by one or more processors, operating characteristics at which a spectral sensor was configured while the spectral sensor captured a multi-dimensional spectral data package of a target object;
identifying, by the one or more processors, one or more artifacts caused by the operating characteristics at which the spectral sensor was configured while the spectral sensor captured the multi-dimensional spectral data package of the target object; and
generating, by the one or more processors, a calibrated multi-dimensional spectral data package of the target object by removing the one or more artifacts from the multi-dimensional spectral data package of the target object.

2. The method of claim 1, wherein the target object is an eye.

3. The method of claim 1, wherein the operating characteristics comprise an optical configuration of the spectral sensor while the spectral sensor captured the multi-dimensional spectral data package of the target object.

4. The method of claim 3, wherein the optical configuration of the spectral sensor comprises one or more properties of one or more optical components operably coupled to the spectral sensor.

5. The method of claim 4, wherein the one or more properties comprise at least one of a focal plane, a focal length, a magnification, a distance between optical components, a working distance, an index of refraction, or a direction of a light path.

6. The method of claim 1, wherein the operating characteristics comprise an illumination power at which the spectral sensor captured the multi-dimensional spectral data package of the target object.

7. The method of claim 1, wherein the operating characteristics comprise:
an optical configuration of the spectral sensor while the spectral sensor captured the multi-dimensional spectral data package of a target object; and
an illumination power at which the spectral sensor captured the multi-dimensional spectral data package of the target object.

8. The method of claim 1, wherein receiving the operating characteristics comprises receiving the operating characteristics from one or more characteristics sensors coupled to one or more optical components operably coupled to the spectral sensor.

9. The method of claim 1, wherein receiving the operating characteristics comprises receiving the operating characteristics from one or more characteristics sensors coupled to one or more adjustable controllers of one or more optical components operably coupled to the spectral sensor.

10. The method of claim 1, wherein receiving the operating characteristics comprises receiving the operating characteristics from one or more characteristics sensors coupled to a light source that illuminates the target object for imaging.

11. The method of claim 1, wherein identifying the one or more artifacts comprises:
identifying, by the one or more processors, a reference multi-dimensional spectral data package captured, by the spectral sensor, of a reference object, wherein the operating characteristics at which the spectral sensor was configured while the spectral sensor captured the reference multi-dimensional spectral data package of the reference object correspond to the operating characteristics at which the spectral sensor was configured while the spectral sensor captured the multi-dimensional spectral data package of the target object; and
identifying, the one or more artifacts in the reference multi-dimensional spectral data package of the reference object.

12. The method of claim 11, wherein generating the calibrated multi-dimensional spectral data package of the target object comprises removing the one or more artifacts identified in the reference multi-dimensional spectral data package of the reference object from the multi-dimensional spectral data package of the target object.

13. A method comprising:
receiving, by one or more processors, a plurality of operating characteristics at which a spectral sensor was configured while the spectral sensor captured a respective plurality of reference multi-dimensional spectral data packages of a reference object;
identifying, by the one or more processors, in each of the plurality of reference multi-dimensional spectral data packages, one or more artifacts caused by each of the plurality of operating characteristics at which the spectral sensor was configured while the spectral sensor captured each of the plurality of the multi-dimensional spectral data package of the reference object;
receiving, by one or more processors, operating characteristics at which the spectral sensor was configured while the spectral sensor captured a multi-dimensional spectral data package of a target object;
selecting, by the one or more processors, from the plurality of reference multi-dimensional spectral data packages, a reference multi-dimensional spectral data package of the reference object, wherein the operating characteristics at which the spectral sensor was configured while the spectral sensor captured the reference multi-dimensional spectral data package of the reference object corresponds to the operating characteristics at which the spectral sensor was configured while the spectral sensor captured the multi-dimensional spectral data package of the target object; and
modifying, by the one or more processors, the multi-dimensional spectral data package of the target object with the reference multi-dimensional spectral data package of the reference object to remove, from the multi-dimensional spectral data package of the target object, one or more artifacts caused by the operating characteristics at which the spectral sensor was configured while the spectral sensor captured the multi-dimensional spectral data package of the target object.

14. The method of claim 13, wherein the target object is an eye.

15. The method of claim 13, wherein the operating characteristics comprise an optical configuration of the spectral sensor while the spectral sensor captured the multi-dimensional spectral data package of the target object and the reference multi-dimensional spectral data package of the reference object.

16. The method of claim 15, wherein the optical configuration of the spectral sensor comprises one or more properties of one or more optical components operatively coupled to the spectral sensor.

17. The method of claim 16, wherein the one or more properties comprise at least one of a focal plane, a focal length, a magnification, a distance between optical components, a working distance, an index of refraction, or a direction of a light path.

18. The method of claim 13, wherein the operating characteristics comprise an illumination power at which the spectral sensor captured the multi-dimensional spectral data package of the target object and the reference multi-dimensional spectral data package of the reference object.

19. A method, comprising:
receiving, by one or more processors, environmental conditions at which a spectral sensor captured a multi-dimensional spectral data package of a target object;
identifying, by the one or more processors, one or more artifacts caused by the environmental conditions at which the spectral sensor captured the multi-dimensional spectral data package of the target object; and
generating, by the one or more processors, a calibrated multi-dimensional spectral data package of the target object by removing the one or more artifacts from the multi-dimensional spectral data package of the target object.

20. A system, comprising:
an illumination source configured to illuminate an object;
a spectral sensor configured to capture a multi-dimensional spectral data package of the object; and
one or more processors, the one or more processors being programmed to:
receive operating characteristics at which the spectral sensor was configured while the spectral sensor captured a multi-dimensional spectral data package of a target object;
identify one or more artifacts caused by the operating characteristics at which the spectral sensor was configured while the spectral sensor captured the multi-dimensional spectral data package of the target object; and
generate a calibrated multi-dimensional spectral data package of the target object by removing the one or more artifacts from the multi-dimensional spectral data package of the target object.

21.-32. (canceled)

33. A system, comprising:
an illumination source configured to illuminate an object;
a spectral sensor configured to capture a multi-dimensional spectral data package of the object; and
one or more processors, the one or more processors being programmed to:
receive a plurality of operating characteristics at which the spectral sensor was configured while the spectral sensor captured a respective plurality of reference multi-dimensional spectral data packages of a reference object;
identify, in each of the plurality of reference multi-dimensional spectral data packages, one or more artifacts caused by each of the plurality of operating characteristics at which the spectral sensor was configured while the spectral sensor captured each of the plurality of the multi-dimensional spectral data package of the reference object;
receive operating characteristics at which the spectral sensor was configured while the spectral sensor captured a multi-dimensional spectral data package of a target object;
select, from the plurality of reference multi-dimensional spectral data packages, a reference multi-dimensional spectral data package of the reference object, wherein the operating characteristics at which the spectral sensor was configured while the spectral sensor captured the reference multi-dimensional spectral data package of the reference object corresponds to the operating characteristics at which the spectral sensor was configured while the spectral sensor captured the multi-dimensional spectral data package of the target object; and
modify the multi-dimensional spectral data package of the target object with the reference multi-dimensional spectral data package of the reference object to remove, from the multi-dimensional spectral data package of the target object, one or more artifacts caused by the operating characteristics at which the spectral sensor was configured while the spectral sensor captured the multi-dimensional spectral data package of the target object.

34.-39. (canceled)
